(19) United States
(12) Patent Application Publication
(10) Pub. No.: US 2023/0270506 A1
(43) Pub. Date: Aug. 31, 2023

(54) SYSTEMS AND METHODS FOR MEDICAL OBJECT TRACKING IN OBSTRUCTED ENVIRONMENTS

(71) Applicant: CAIRA Surgical, New York, NY (US)

(72) Inventors: Gordon L. GOODCHILD, West Palm Harbor, FL (US); Morteza MEFTAH, New York, NY (US)

(21) Appl. No.: 18/124,554

(22) Filed: Mar. 21, 2023

Related U.S. Application Data
(63) Continuation-in-part of application No. 18/069,338, filed on Dec. 21, 2022.
Application No. 18/069,338 is a continuation-in-part of application No. 17/901,475, filed on Sep. 1, 2022.
Application No. 17/901,475 is a continuation of application No. 17/017,015, filed on Sep. 10, 2020, now Pat. No. 11,432,882.
Application No. 17/017,015 is a continuation-in-part of application No. 16/573,095, filed on Sep. 17, 2019.

(51) Int. Cl. A61B 34/20

(52) U.S. Cl. CPC A61B 34/20; A61B 2090/3916

(57) ABSTRACT

A system for alerting a medical provider of a possible displacement of a beacon includes the beacon, a control device, and a transceiver. The beacon includes sensors (for example, but not limited to, an IMU and a frequency shifter) and at least one antenna that exchanges signals with the transceiver. The IMU and the frequency shifter are used in a coordinated way to alert the medical provider if the beacon has been displaced in a way that might impact the medical procedure. As the beacon moves, when an anomalous correlation between the change over time of the IMU position data (of the beacon) and the frequency shifter position data (of the beacon) is detected, and the beacon has undergone a gravity/inertial moment (according to the IMU data) that exceeds a pre-selected threshold, the medical provider is alerted that the beacon's position might have shifted enough to impact the medical procedure.

### CROSS-REFERENCE TO RELATED APPLICATIONS

This application is a continuation-in-part of U.S. application Ser. No. 18/069,338 filed Dec. 21, 2022, which is a continuation in part of U.S. application Ser. No. 17/901,475, filed Sep. 1, 2022, which is a continuation of U.S. application Ser. No. 17/017,015 filed Sep. 10, 2020, now issued as U.S. Pat. No. 11,432,882, which is a continuation-in-part and claims priority to U.S. patent application Ser. No. 16/573,095, filed on Sep. 17, 2019, entitled SYSTEM AND METHOD FOR MEDICAL OBJECT TRACKING. This application is related to U.S. Provisional Patent Application Serial No. ______, filed on Mar. 21, 2023, entitled MAGNETIC BASE WITH LOCK FEATURE. The entirety of all of these documents is incorporated herein by reference.

### FIELD

Systems and methods consistent with this disclosure relate to location monitoring of objects in a medical environment. More particularly, this disclosure relates to location monitoring in medical environments that include physical and/or electronic obstructions.

### BACKGROUND

Placement of implants in bones or soft tissue requires precise planning. For example, in joint replacement orthopedic surgery, precise boney cuts are essential to achieve optimum outcomes. Historically, to achieve this, manual cutting blocks that reference bony landmarks, limb anatomical alignment, and visual cues have been designed to help the surgeon place appropriate guides; however, these guides lack the necessary precision due to issues inherent to manual cutting jigs.

In recent years, computer-assisted surgery (CAS) has been used to improve the accuracy of implant positioning. Existing CAS systems can require optical trackers for the computer to identify bones that are in constant movement during surgery. These optical trackers include multiple large pins that need to be fixed into each bone, most of the time through separate incisions, that may cause fractures and more pain for the patients. Further, these optical trackers can require bulky optical apparatus that require an unobstructed line of sight for a camera, and a large amount of hardware and software to operate. Moreover, currently there is no systematic way to adjust the implant position based on a patient's individual soft-tissue tension. Most CAS are tailored to achieve a “balanced” soft tissue tension by surgeons' manual tests. These manual techniques are not accurate or reproducible since human anatomy varies.

Radar technology can use continuous wave RF waveform generation at various frequencies to track the distance and speed of an object based on the return of the signal and its modified frequency. An object traveling away from a radar source, for example, will return a longer time delay at each detection, and an object traveling towards the source will return a shorter time delay at each detection. Currently, there are radar applications available in the automobile and defense industries that aim to achieve high precision location tracking. One such radar module is commercially available, operating at 77 GHZ with wide 4 GHZ bandwidth that allows for high resolution and accuracy with the use of frequency modulate continuous wave (FMCW) radar. However, currently there are no applications available to achieve a resolution below one millimeter in short range.

As technological advances in tracking, location monitoring, and navigation have occurred, an operating room (OR) environment that seeks to take advantage of these advances tends to require ever-growing amounts of equipment and electronics. Each of these tendencies raise further issues. For example, the presence of additional equipment in an OR environment can raise a concern regarding the maintenance of a sterile environment. One of the simplest solutions to the maintenance of a sterile environment is to use sterile draping over OR equipment and objects. However, draping can degrade or prevent the operation, or otherwise obstruct the utility of a tracking, location monitoring, and/or navigation system—such as optics-based or computer-vision systems that require an unobstructed line-of-sight, with which drapery and other objects interfere. Further still, the presence of additional electronics in an OR environment typically increases the radio frequency (RF) “noise” in the environment, which can also interfere with or otherwise obstruct the effectiveness of tracking, location monitoring, and navigation systems.

Therefore, existing systems suffer from one or more issues.

### SUMMARY

The techniques of this disclosure generally relate to object location monitoring in a medical environment. More particularly, this disclosure relates to location monitoring in medical environments that include physical and/or electronic obstructions.

In one aspect, embodiments consistent with the present disclosure include a system for radio-frequency-based location determination in a draped environment. Consistent with this disclosure, the system can include a control device with a processor and a memory, where the memory includes a non-transitory computer readable medium for storing instructions that when executed by the processor cause the processor to perform a method for location determination, where the method for location determination includes generating transmission instructions from the control device and analyzing received data received by the control device. Consistent with this disclosure, the system can also include a plurality of radio frequency transceivers in communication with the control device, where each of the plurality of radio frequency transceivers is configured to emit a radio frequency signal at a respective frequency value responsive to the transmission instructions from said control device. Consistent with this disclosure, the system can also include at least one active beacon, where the at least one active beacon is configured to transmit a modified radio frequency signal at a respective beacon frequency value responsive to receipt of the radio frequency signal from any of the plurality of radio frequency transceivers at any of the respective frequency values. Consistent with this disclosure, the respective beacon frequency value can be shifted by a first amount from the respective frequency value of the radio frequency signal received at the active beacon. In an embodiment consistent with this disclosure, each of the plurality of radio frequency transceivers can be configured to send the received data to the control device responsive to receipt of the modified radio frequency signal from the at least one active beacon, where the received data includes data based on the modified radio frequency signal. Furthermore, in an embodiment, the control device including the processor and the memory can be configured to determine a location of the at least one active beacon based upon the transmission instructions and the received data. Further still, in an embodiment, the draped environment can include draping material between at least one of the plurality of radio frequency transceivers and the at least one active beacon, where the draping material, each of the respective frequency values, and each of the respective beacon frequency values are selected such that the draping material is substantially transparent to the emitted radio frequency signals and substantially transparent to the modified radio frequency signals. Moreover, in an embodiment, each of the plurality of radio frequency transceivers can be in a fixed spatial relationship to each other.

In a further aspect, a system consistent with the present disclosure can include any of the above embodiments, and further include a plurality of active beacons configured to transmit a plurality of modified radio frequency signals at a respective individual beacon frequency value responsive to receipt of the radio frequency signal from any of the plurality of radio frequency transceivers at any of the respective frequency values. In such a further aspect, the respective second beacon frequency value is shifted by a second amount from the respective frequency value of the radio frequency signal received at the second active beacon. Further, in an embodiment, the second amount is different from said first amount, each of the plurality of radio frequency transceivers is configured to send received second data to the control device responsive to receipt of the second modified radio frequency signal from the second active beacon, and the control device, including the processor and the memory, is configured to determine a location of the second active beacon based upon the transmission instructions and the received second data. Further still, in an embodiment, the draping material and each of the respective second beacon frequency values are selected such that the draping material is substantially transparent to the second modified radio frequency signals.

In a further aspect, a system consistent with the present disclosure can include any of the above embodiments where the modified radio frequency signal at a respective beacon frequency value is a first Doppler-shifted signal, the second modified radio frequency signal at a respective second beacon frequency value is a second Doppler-shifted signal, the third modified radio frequency signal at a respective third beacon frequency value is a third Doppler-shifted signal, and so on.

In another aspect, a system consistent with the present disclosure can include any of the above embodiments where the control device, including the processor and the memory, is configured to use range-Doppler processing for the determination of the locations of a plurality of beacons.

In a further aspect, a system consistent with the present disclosure can include any of the above embodiments where the control device, including the processor and the memory, is configured to determine an orientation of the at least one active beacon relative to the second active beacon.

In another aspect, a system consistent with the present disclosure can include any of the above embodiments where the draped environment is a surgical environment, where the location of the at least one active beacon is an absolute location value of the at least one active beacon in the surgical environment, where the location of the second active beacon is an absolute location value of the second active beacon in the surgical environment, and where the orientation is an absolute orientation value within the surgical environment.

In a further aspect, a system consistent with the present disclosure can include any of the above embodiments further including a display device. In an aspect consistent with the present disclosure, the absolute location value of the at least one active beacon, the absolute location value of the second active beacon, the absolute orientation value, and the surgical environment are depicted in a representation on the display device.

In an additional aspect, a system consistent with the current disclosure can include any of the above embodiments further including a storage device, where the absolute location value of the at least one active beacon, the absolute location value of the second active beacon, and the absolute orientation value are stored in the storage device.

Further still, in an aspect, a system consistent with the current disclosure can include any of the above embodiments where the at least one active beacon is removably attachable to a medical object, and where the system for radio-frequency-based location determination is a system for medical object tracking.

Moreover, in an aspect, a system consistent with the current disclosure can include any of the above embodiments where the at least one active beacon comprises a reflector configured to reflect the radio frequency signal at the respective frequency value, where the system for radio-frequency-based location determination is configured to detect the reflected radio signal, and where the system is configured to calibrate the control device for location determination based, at least in part, on the detected reflected radio signal.

In an additional aspect, a method for radio-frequency-based location determination in a draped environment consistent with the present disclosure can include generating radio frequency transmission instructions from a control device, the transmission instructions being communicated to at least three radio frequency transceivers. The method can further include emitting at least three radio frequency signals from the three radio frequency transceivers responsive to the transmission instructions, each radio frequency transceiver of the three radio frequency transceivers emitting a respective radio frequency signal at a respective frequency value such that the three radio frequency signals are emitted at three respective frequency values. Consistent with this disclosure, the method can further include: (1) receiving a first modified radio frequency signal from an active beacon, the first modified radio frequency signal being a frequency-shifted re-transmission of a first of the three emitted radio frequency signals at a first of the three respective frequency values; (2) receiving a second modified radio frequency signal from the active beacon, the second modified radio frequency signal being a frequency-shifted re-transmission of a second of the three emitted radio frequency signals at a second of the three respective frequency values; and (3) receiving a third modified radio frequency signal from the active beacon, the third modified radio frequency signal being a frequency-shifted re-transmission of an other of the three emitted radio frequency signals at an other of the three respective frequency values. In an aspect, the method can further include generating data from the received first modified radio frequency signal, the received second modified radio frequency signal, and the received third modified radio frequency signal, and transmitting the generated data to the control device, and analyzing the generated data received at the control device to determine location data for the active beacon. Consistent with the disclosure, the draped environment can include draping material between at least one of the three radio frequency transceivers and the active beacon, where the draping material, each of the three respective frequency values, and each frequency value of the frequency-shifted re-transmissions are selected such that the draping material is substantially transparent to the emitted radio frequency signals and substantially transparent to the modified radio frequency signals. Further still, consistent with this disclosure, each of the three radio frequency transceivers can be in a fixed spatial relationship to each other.

In a further aspect, a method consistent with this disclosure can include the previous embodiment, where the first modified radio frequency signal from the active beacon is a first Doppler-shifted signal, the second modified radio frequency signal from the active beacon is a second Doppler-shifted signal, and the third modified radio frequency signal from the active beacon is a third Doppler-shifted signal.

In an additional aspect, a method consistent with the present disclosure can include any of the previous method embodiments where the determination of the location data for the active beacon is performed using range-Doppler processing.

Further still, in an aspect, a method consistent with the present disclosure can include any of the previous method embodiments, further including: analyzing the generated data received at the control device to determine orientation data for the active beacon.

Moreover, in an aspect, a method consistent with the present disclosure can include any of the previous method embodiments, where the draped environment is a surgical environment, where the active beacon is removably attachable to a medical object, and where the method for radio-frequency-based location determination is a method for medical object tracking.

In an additional aspect, an embodiment consistent with the present disclosure can include a non-transitory computer readable medium storing instructions that when executed by a processor in a control device cause the processor to perform a method for radio-frequency-based location determination in a draped environment. In an aspect, the method can include: generating radio frequency transmission instructions, the transmission instructions being communicated from the control device to at least three radio frequency transceivers, where at least three radio frequency signals from the three radio frequency transceivers responsive to the transmission instructions are emitted, each radio frequency transceiver of the three radio frequency transceivers emitting a respective radio frequency signal at a respective frequency value such that the three radio frequency signals are emitted at three respective frequency values. Consistent with this disclosure, the method can further include receiving generated data from the three radio frequency transceivers, the generated data being data generated from: (1) a first modified radio frequency signal received from an active beacon, the first modified radio frequency signal being a frequency-shifted re-transmission of a first of the three emitted radio frequency signals at a first of the three respective frequency values; (2) a second modified radio frequency signal received from the active beacon, the second modified radio frequency signal being a frequency-shifted re-transmission of a second of the three emitted radio frequency signals at a second of the three respective frequency values; and (3) a third modified radio frequency signal received from the active beacon, the third modified radio frequency signal being a frequency-shifted re-transmission of an other of the three emitted radio frequency signals at an other of the three respective frequency values. Consistent with this disclosure, the method can also include analyzing the generated data received at the control device to determine location data for the active beacon, where the draped environment comprises draping material between at least one of the three radio frequency transceivers and the active beacon, where the draping material, each of the three respective frequency values, and each frequency value of the frequency-shifted re-transmissions are selected such that the draping material is substantially transparent to the emitted radio frequency signals and substantially transparent to the modified radio frequency signals, and where each of the three radio frequency transceivers are in a fixed spatial relationship to each other.

In a further aspect, a non-transitory computer readable medium storing instructions consistent with this disclosure can include the previous instructions where the first modified radio frequency signal from the active beacon is a first Doppler-shifted signal, the second modified radio frequency signal from the active beacon is a second Doppler-shifted signal, and the third modified radio frequency signal from the active beacon is a third Doppler-shifted signal.

Further still, in an aspect, a non-transitory computer readable medium storing instructions consistent with this disclosure can include any of the previous instructions where the determination of the location data for the active beacon is performed by the processor using range-Doppler processing.

In another aspect, a non-transitory computer readable medium storing instructions consistent with this disclosure can include any of the previous instructions where the method further includes: analyzing the generated data received at the control device to determine orientation data for the active beacon.

Moreover, in a further aspect a non-transitory computer readable medium storing instructions consistent with this disclosure can include any of the previous instructions where the draped environment is a surgical environment, where the active beacon is removably attachable to a medical object, and where the method for radio-frequency-based location determination is a method for medical object tracking.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method for detecting possible inaccurate information associated with a medical object during surgery. The method also includes registering a plurality of sensors to a coordinate frame, the plurality of sensors being associated with at least one beacon, the beacon being associated with the medical object; calibrating, possibly substantially continuously, a first sensor of the plurality of sensors using information from a second sensor of the plurality of sensors as the at least one beacon moves; collecting first sensor position data in the coordinate frame of the at least one beacon from the first sensor and second sensor position data in the coordinate frame of the at least one beacon from the second sensor; and raising an alert when a change over time of a correlation between the first sensor position data and the second sensor position data exceeds a first threshold, and when one of the plurality of sensors detects an inertial moment that exceeds a second threshold. Other embodiments of this aspect include corresponding computer systems, apparatus, and computer programs recorded on one or more computer storage devices, each configured to perform the actions of the methods.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method for detecting possibly inaccurate information associated with a medical object during surgery. The method also includes registering a plurality of sensors to a coordinate frame, the plurality of sensors being associated with at least one beacon, the beacon being associated with the medical object; calibrating, possibly continuously, a first sensor of the plurality of sensors using information from a second sensor of the plurality of sensors as the at least one beacon moves; collecting first sensor position data in the coordinate frame of the at least one beacon from the first sensor and second sensor position data in the coordinate frame of the at least one beacon from the second sensor; and raising an alert when a change over time of a correlation between the first sensor position data and the second sensor position data exceeds a first threshold, and when one of the plurality of sensors detects an inertial moment that exceeds a second threshold. Other embodiments of this aspect include corresponding computer systems, apparatus, and computer programs recorded on one or more computer storage devices, each configured to perform the actions of the methods.

Implementations may include one or more of the following features. The method as where the at least one beacon may include: a power supply configured to power the plurality of sensors. The arm may include: an anatomically-consistent geometric shape. The first sensor may include: an inertial measurement unit (IMU) configured to provide IMU beacon location data and the inertial moment of the at least one beacon to at least one controller. The second sensor may include: a frequency shifter configured to receive a signal from an rf transceiver and to provide a signal to at least one controller. The method may include: determining a beacon location of the at least one beacon using range-doppler processing. The method as may include: receiving data from the first sensor wirelessly. The method as may include: correcting drift error of the first sensor using the second sensor. The method as may include: calibrating, possibly continuously, the first sensor based on data from a plurality of the second sensors. Implementations of the described techniques may include hardware, a method or process, or computer software on a computer-accessible medium.

One general aspect includes a system for detecting possible inaccurate information associated with a medical object during surgery. The system also includes at least one beacon including a plurality of sensors; an anchor configured to operably couple with the medical object; an arm having a first end and a second end, the first end configured to operably couple with the at least one beacon, the second end configured to operably couple with the anchor; and at least one controller communicatively coupled with at least one frequency shifter, the at least one frequency shifter being configured to transmit a first signal to the at least one controller and receive a second signal from at least one transceiver, the at least one controller configured to execute instructions including: registering the plurality of sensors to a coordinate frame, the plurality of sensors being associated with the at least one beacon, the at least one beacon being associated with the medical object; calibrating, possibly continuously, a first sensor of the plurality of sensors, the calibrating using information from a second sensor of the plurality of sensors as the at least one beacon moves; collecting first sensor position data in the coordinate frame of the at least one beacon from the first sensor and second sensor position data in the coordinate frame of the at least one beacon from the second sensor; and raising an alert if a change over time of a correlation between the first sensor position data and the second sensor position data exceeds a first threshold, and if one of the plurality of sensors detects an inertial moment that exceeds a second threshold. Other embodiments of this aspect include corresponding computer systems, apparatus, and computer programs recorded on one or more computer storage devices, each configured to perform the actions of the methods.

Implementations may include one or more of the following features. The system as where the at least one beacon may include: a power supply configured to power the plurality of sensors. The arm may include: an anatomically-consistent geometric shape. The first sensor may include: an inertial measurement unit (IMU) configured to provide IMU beacon location data and the inertial moment of the at least one beacon to the at least one controller. The second sensor may include: a positional tracking modality. The at least one controller may include: communicative coupling to the at least one transceiver. The anchor may include: a plurality of teeth configured to operably connect the anchor with the medical object. The plurality of teeth may include: a plurality of triangle-shaped forms operably coupled with the anchor, and a one or more component orthogonally coupled with the plurality of triangle-shaped forms. The at least one controller is configured to use range-doppler processing to determine a location of the at least one beacon. The alert is a warning indicator on user interface screen. The user interface screen may include: an association with at least one of a laptop computer device, a handheld computer device, a desktop computer device, and a tablet computer device. Implementations of the described techniques may include hardware, a method or process, or computer software on a computer-accessible medium.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a diagram of an example system according to one or more embodiments of the invention;

FIG. 2 is block diagram of FIG. 1 according to one or more embodiments of the invention;

FIG. 3 is a flow diagram of an example process according to one or more embodiments of the disclosure;

FIG. 4 is a flow diagram of an example process according to one or more embodiments of the disclosure;

FIG. 5 is an exploded view of an exemplary beacon constructed according to one or more embodiments of the disclosure;

FIG. 6 is an assembled view of the beacon shown in FIG. 5;

FIG. 7 is view showing three beacons mounted to medical objects, namely, the femur, the tibia, and a cutting element of robotic arm;

FIG. 8 depicts an embodiment of an apparatus for mounting RF transceivers in a “constellation” configuration consistent with this disclosure;

FIG. 9 depicts an example of the apparatus of FIG. 8 attached to an operating table, consistent with this disclosure;

FIG. 10 depicts an example of the operating table and apparatus of FIG. 9 draped with sterile coverings, consistent with this disclosure;

FIG. 11 depicts another embodiment of an apparatus for mounting RF transceivers in a “constellation” configuration consistent with this disclosure;

FIG. 12 depicts an example of a support apparatus for RF transceivers that attaches to a rolling stand, consistent with this disclosure;

FIGS. 13A and 13B depict an example of the stand and support apparatus of FIG. 12 draped with sterile coverings, and supporting a frame surrounding a monitor, consistent with this disclosure;

FIG. 14 depicts an exemplary geometry for trilateration consistent with this disclosure;

FIG. 15 is a schematic block diagram of the system consistent with this disclosure;

FIG. 16 shows pictorial representations of a configuration of an anchor and an arm consistent with this disclosure;

FIG. 17 is a pictorial representation of a configuration of a device consistent with this disclosure attached to an anatomical feature;

FIG. 18 shows pictorial representations of devices consistent with this disclosure attached to two components of an anatomical feature;

FIG. 19 shows perspective side and bottom views of the communications and sensor device consistent with this disclosure;

FIG. 20A shows perspective, plan, and side views of the anchor consistent with this disclosure;

FIG. 20B shows a cross-section of the anchor consistent with this disclosure

FIG. 21 shows photographic views of the anchor consistent with this disclosure placed in the vicinity of a spacer block and other surgery apparatus;

FIG. 22 shows photographic views of the arms and beacons consistent with this disclosure attached to the anchor; and

FIG. 23 is a flowchart of a method consistent with this disclosure.

### DETAILED DESCRIPTION

Exemplary embodiments described herein include combinations of apparatus components and processing steps related to object location monitoring. Components have been represented where appropriate by conventional symbols in the drawings, showing only those specific details that are pertinent to understanding the embodiments so as not to obscure the disclosure with details that will be readily apparent to those of ordinary skill in the art having the benefit of the description herein. Like numbers refer to like elements throughout the description.

As used herein, relational terms, such as “first” and “second,” “top” and “bottom,” and the like, may be used solely to distinguish one entity or element from another entity or element without necessarily requiring or implying any physical or logical relationship or order between such entities or elements. The terminology used herein is for the purpose of describing particular embodiments only and is not intended to be limiting of the concepts described herein. As used herein, the singular forms “a”, “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof.

In embodiments described herein, the joining term, “in communication with” and the like, may be used to indicate electrical or data communication, which may be accomplished by physical contact, induction, electromagnetic radiation, radio signaling, infrared signaling or optical signaling, for example. One having ordinary skill in the art will appreciate that multiple components may interoperate, and modifications and variations are possible of achieving the electrical and data communication.

In some embodiments described herein, the term “coupled,” “connected,” and the like, may be used herein to indicate a connection, although not necessarily directly, and may include wired and/or wireless connections.

Unless otherwise defined, all terms (including technical and scientific terms) used herein have the same meaning as commonly understood by one of ordinary skills in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

In accordance with various embodiments disclosed herein, in an OR, radar sources (e.g., RF transceivers) that can “see through” common OR obstructions can be used to trilaterate the location of an object that returns RF waves more efficiently than the surrounding objects. Furthermore, varying the frequency of the waves emitted by the RF sources can allow the positioning of objects to be isolated more easily and tracked more precisely compared to conventional optical tracking systems. For some embodiments where an object's positional accuracy and precision is required to be submillimeter, the systems described herein may use RF waves of multiple wavelengths to determine the object's position, so that the object's location is not misjudged if it is in between the wavelengths. In some implementations, submillimeter accuracy may be defined as 1 mm or less accuracy, such as 1 μm-1 mm accuracy (micrometer-millimeter).

Techniques, systems, and computer-readable media disclosed herein relate to systems for the precise tracking of an object in an area of interest. The techniques and systems can include one or more radio frequency (RF) transceivers, which may also be referred to as radar transceivers. A plurality of RF transceivers operating as transmitters and receivers at fixed locations relative to each other will be referred to herein as a “constellation” of transceivers.

As used herein, transceivers that are at “fixed” locations relative to each other are not necessarily at fixed locations in a larger environment. By way of example, only, a surgical operating bed or table may be configured to exhibit mobility within an OR; however, a constellation of transceivers, consistent with this disclosure, can be fixed to a frame or other apparatus that, in turn, is fixed to the surgical operating bed or table. In this way, a constellation of transceivers can be configured to be at fixed locations relative to each other (as well as at fixed locations relative to the surgical operating bed or table), but, nonetheless, can be configured to exhibit mobility within the OR region as a whole.

Further still, as used herein, transceivers that are at “fixed” locations relative to each other during a surgical operation are not necessarily at the same “fixed” locations at all times and during other surgical operations. Again, by way of example only, during a surgical operation on a first patient, three transceivers in a first constellation configuration can be configured to lie in a plane that, itself, exhibits an angle or a skew, such as a 60° skew, relative to a plane defined by a surgical operating table or bed on which the first patient rests. Furthermore, relative to a narrow surgical region of interest, the three transceivers can be configured to lie in an arc, with the first and second transceivers separated by 45°, and the second and third transceivers separated by 45° (with the first and third transceivers necessarily separated by 90°) with respect to the triangle formed by the three transceivers. During a subsequent surgical operation on a second patient, the same three transceivers in a second constellation configuration can be configured to lie in a different plane that exhibits (for example) a 90° angle or skew relative to a plane defined by a surgical operating table or bed on which the second patient rests. Furthermore, relative to a different, narrow, surgical region of interest, the three transceivers can be configured to lie in an arc, with the first and second transceivers separated by 60°, and the second and third transceivers separated by 60° (with the first and third transceivers necessarily separated by 120°). Consistent with embodiments disclosed herein, a tracking system can be “preset” to operate according to both the first constellation configuration at a first time, and the second constellation configuration at a second time. Furthermore, one of ordinary skill in the art will appreciate that any number of “preset” constellation configurations (with varying skew angles, varying arc separations, and varying numbers of transceivers) can be accommodated consistent with this disclosure.

As disclosed herein, each of the transceivers can be configured to transmit RF signals at a particular, distinct RF frequency. Techniques and systems disclosed herein can also include one or more active beacons. As disclosed herein, an active beacon can be configured to be responsive to incoming RF signals from the plurality of transceivers discussed above. Specifically, upon receipt of an incoming RF signal from one of the plurality of transceivers, an active beacon consistent with this disclosure can be configured to emit an RF signal that is modified relative to the RF signal it receives. More specifically, consistent with this disclosure, the modified RF signal emitted by an active beacon (i.e., the modified outgoing RF signal) can be a signal at a RF frequency that is shifted relative to the incoming RF signal received by the active beacon. Such a modified signal may be referred to herein as a “frequency-shifted modified RF signal.” Further still, the frequency-shifted modified RF signal emitted by an active beacon can be configured to transmit over short- to mid-range distances. As used herein, short-to mid-range distances can encompass the sterile field portion associated with a conventional surgical environment, such as the sterile field area within a typical OR. For example, and without limitation, an exemplary distance between radar and beacon can be between 3 feet and 5 feet, representing a sterile field portion of a typical OR, where a typical OR can be 400 to 600 square feet.

Accordingly, consistent with this disclosure, active beacons disclosed herein can be configured to actively re-transmit the frequency-shifted RF signals upon receipt of, or in response to, the RF signals from a plurality of transceivers. Each active beacon can be configured to re-transmit at a unique or distinct frequency-shifted value that is different from each of the other active beacons in use during an operation or surgical procedure.

Consistent with techniques and systems disclosed herein, a control device, in communication with the plurality of transceivers, can be configured to provide transmission instructions to each of the plurality of transceivers. Each of the transceivers, in turn, can be configured to transmit an RF signal at a unique (e.g., distinct with respect to the other transceivers) frequency into the surgical environment, responsive to the transmission instructions from the control device. Moreover, because each of the transceivers can be configured to transmit at a unique RF frequency into the surgical environment, and each of the active beacons can be configured to re-transmit a unique (e.g., distinct with respect to the other active beacons) frequency-shifted RF signal back into the surgical environment based on the RF signal it receives, the control device (together with each of the transceivers) can be configured to recognize each re-transmitted signal from a single active beacon, where each re-transmitted signal is based upon and/or responsive to one of the independently-emitted RF signals from the plurality of transceivers in the constellation.

In various embodiments, based upon this information (i.e., the time of the original transmission from each of the transceivers, the time at which the re-transmitted frequency-shifted signal is received back at each of the transceivers, and the fixed location of each of the transceivers in the constellation), the control device determines a location of the active beacon relative to the constellation by trilateration. For example, in a system consisting of three transceivers (T1, T2, T3) and one active beacon (A), where the distance between each of the transceivers is fixed and known (i.e., the distances T1T2, T1T3, and T2T3), the transmission/re-transmission information associated with each transceiver and the active beacon A is used to calculate the distance between each transceiver and the active beacon (i.e., the distances T1A, T2A, and T3A). Accordingly, the control device can be configured to recognize that there are three different triangles formed between any two pairs of the transceivers and the active beacon A (i.e., the triangles formed with the vertices: (T1, T2, A), (T1, T3, A), and (T2, T3, A)). Based upon this information, trilateration techniques can be used to determine the position of the active beacon A relative to the constellation in three-dimensional space.

Moreover, because each active beacon can be configured to re-transmit at a unique/distinct frequency-shifted value, the re-transmitted signals from each active beacon can be independently recognized, and so the location of each active beacon relative to the constellation in three-dimensional space can be separately determined.

Furthermore, because the re-transmitted signals are frequency-shifted values, a set of re-transmitted RF signals from one active beacon can correspond to a unique or distinct “Doppler shift” set of signals from the plurality of transceivers, creating a pseudo-velocity profile for the active beacon that is well beyond clutter noise limits. This can create an isolated environment for each active beacon within an environment's “Doppler” map. Additionally, these active beacons: (1) can be designed to achieve accuracy and precision required for surgical tracking; (2) can be configured, through signal amplification, to increase the signal to noise ratio; (3) can exhibit a small footprint; (4) can be disposable; and (5) can be used with off-the-shelf batteries.

Consistent with this disclosure, active beacon trilateration (e.g., as described above) can be used for navigational tracking of medical objects, such as anatomical parts and surgical instruments. Examples can include bone tracking for orthopedic applications and tool tracking, such as tracking a bone saw, robotic arm or robotic end effector for orthopedic applications. All transceivers can be configured to emit RF waves at varying frequencies in continuous waves or pulses of microseconds. For example, and without limitation, RF signal generation in a transceiver consistent with this disclosure can be digitally synthesized, which allows each transceiver to generate an RF signal with distinguishable, or unique characteristics. Moreover, each active beacon can be configured to introduce a distinguishable variation (or shift) from each other beacon. Furthermore, each returning or responsive wave (provided by each active beacon) provides scene, or environmental, information with the encoded different frequencies from the active beacons. The scene, or environmental, information from the responsive waves can be used to generate a Range/Doppler map. Given the calibrated (or fixed) locations of the transceivers with respect to each other within a known coordinate system (i.e., a constellation configuration), and given that the system with fixed transceivers can be provided with a “range zero” calibration (i.e., initial calibration) to account for any range inaccuracies, each active beacon can be accurately tracked in three-dimensional space during the duration of the tracking frame or a known range can be established to account for any range inaccuracies. In this manner, each active beacon can be accurately tracked in three-dimensions during the duration of a tracking frame.

In one or more embodiments, the systems described herein improve over existing optical systems and simplify the tracking of medical objects, such as bones, using radar-wave-based technology (i.e., RF signals) that can penetrate through obstructions that impede the operation of conventional, optical-based surgical tracking systems, such as obstructions made of cloth, fabric, paper, plastic, and glass, among other things. The radar-wave-based systems described herein can allow a surgeon to disrupt, either fully or partially, the direct line-of-sight between the transceiver(s) and the beacon(s) without loss of signal, which can increase the safety of the surgery, as the system is able to track the objects despite the line-of-sight disruption. Although an obstruction may cause an undesirable drop in signal strength, the systems described herein can address this by being configured to operate with additional (e.g., more than three) transceivers (which will increase the available data for trilateration calculations) and can maintain tracking and accuracy provided that a minimum of three transceivers are configured to interact with (e.g., receive a usable signal from), and thereby detect, all active beacons of interest.

In various embodiments, a set of transceivers, static or moving, can be configured to emit RF signals in the area of interest. The area of interest can contain a set of active RF beacons, each configured to re-radiate a unique signal back to the transceivers so that the three-dimensional position of each active RF beacon can be determined through signal processing and calculations, such as trilateration. In some embodiments, the system may also determine the inclination or orientation of each beacon, for example, based on data from sensors in each beacon.

In various embodiments, the RF beacons, which are typically disseminated in the area of interest, can be configured to receive the RF signals from the transceivers, shift the frequencies of the incident RF signal within prescribed values, and actively re-transmit the frequency-shifted RF signal. Each beacon can be configured to impose a unique frequency shift to the incoming RF signal relative to the frequency shifts of the other RF beacon(s), thus permitting its identification after signal processing. Specifically, these beacon-generated frequency shifts can be processed by a control device coupled to the transceivers in a manner similar to radar targets exhibiting specific Doppler-shifted frequency values.

In various embodiments, through known Range-Doppler processing, or similar Moving-Target-Indication techniques, the control device can be configured to determine the range (e.g., the distance from a given transceiver) and Doppler shift of each of the beacon signals and echoes in the area of interest. Echoes exhibiting zero or near-zero Doppler values can correspond to clutter, i.e. environmental or human, and can be removed by a signal processor. “Echoes” that are in fact signals from beacons corresponding to specific Doppler frequencies associated with active beacon(s) in the area of interest can also be isolated, processed and tracked. The three-dimensional location of each RF beacon can be determined through trilateration of the range information collected across all the transceivers radiating within the area of interest.

In some embodiments, an RF beacon may include or be coupled with an accelerometer(s), which provides data describing the orientation of the RF beacon to the system, for example, via signals to a transceivers. In such embodiments, the control device can further be configured to calculate or determine both position and orientation information of an object, for example, a bone, to which an accelerometer-equipped RF beacon is affixed. The motion of such an object can be determined and tracked using the RF transceivers as described herein, and can be displayed on a display device that is in communication with the control device, to allow a user to manipulate the object based on its defined location with external tools. In some embodiments, the control device, coupled with the display device, can be configured with augmented reality (such as using a virtual reality representation of the area of interest, overlaid on the tracked object) to allow the user to view the user's manipulation of the object on the display.

According to one aspect of the invention, a system for medical object tracking is provided. The system can include a plurality of radio frequency transceivers where each of the plurality of radio frequency transceivers can be configured to emit a radio frequency signal at a respective frequency. The system can further include a radio frequency active beacon removably attached to a medical object, which may be a boney structure, where the radio frequency active beacon is configured to actively (or passively, in some instances) modify the radio frequency signals from the plurality of radio frequency transceivers. The system can further include a control device in communication with the plurality of radio frequency transceivers where the control device includes processing circuitry and/or software code (implemented by a processor and memory) configured to determine a location of the medical object in three-dimensional space based at least in part on the re-transmitted, modified radio frequency signals.

In one embodiment, six degrees of freedom and tilt measurement can be obtained through the use of one or more accelerometers and/or multiple re-radiating antennas associated with a single active beacon (i.e., two or more re-radiating antennas affixed to a single beacon) and/or multiple active beacons (each with a single re-radiating antenna). More specifically, multiple re-radiating antennas can be placed on the same beacon or multiple separate beacons (each with a single re-radiating antenna) can be placed in fixed positions on the bone or other object to be tracked.

In instances where a single beacon is configured with more than a single re-radiating antenna, consistent with this disclosure, each separate re-radiating antenna can be configured to re-radiate at a different frequency-shifted frequency relative to the other antennae in order to provide a distinct, re-radiated, frequency-shifted RF signal from each antenna of the beacon.

According to one or more embodiments, the radio frequency beacon includes a conical (or horn) component or other antenna, the conical (or horn) component or antenna configured to re-radiate radio frequency signals. According to one or more embodiments, the plurality of radio frequency transceivers can be configured to interrogate a respective predefined area at a predefined sweep frequency. The control device can be configured to modify the respective predefined area and predefined sweep frequency based at least in part on the location of the medical object.

According to one or more embodiments, the medical object is one of a surface of a bone and a medical device. According to one or more embodiments, the determination of the location of a reference point on the medical object in three-dimensional space includes determining, for each respective re-transmitted radio frequency signal, a respective location in three-dimensional space of the reference point on the medical object. The determined location of the reference point of the medical object in three-dimensional space can be based on the determined respective locations in three-dimensional space of one or more active beacons and their known affixed positions on the medical object.

According to one or more embodiments, the radio frequency beacon can include at least one accelerometer configured to generate accelerometer data. At least one of the re-transmitted radio frequency signals can include the accelerometer data. According to one or more embodiments, the control device can be further configured to determine an orientation of the radio frequency beacon in three-dimensional space based at least in part on the accelerometer data. According to further embodiments, the control device can be configured to determine an orientation of a radio frequency beacon with multiple antennae in three-dimensional space based at least in part on trilateration data associated with each separate antenna.

According to another aspect of the invention, a method implemented in a system for medical object tracking is provided. A radio frequency signal is emitted at a respective frequency by each radio frequency transceiver of a plurality of radio frequency transceivers. The radio frequency signals can then be re-transmitted by a radio frequency beacon removably attachable to the medical object, to be received at the plurality of radio frequency transceivers. The re-transmitted signals may be frequency-shifted signals that are emitted by the radio frequency beacon. The radio frequency beacon may be affixed to a medical object, and a location of the medical object in three-dimensional space is determined based at least in part on the re-transmitted radio frequency signal.

According to one or more embodiments, the re-transmitted radio frequency signals can be emitted by a conical (or horn) component of the radio frequency beacon and/or an antenna. According to one or more embodiments, the emitting, at each radio frequency transceivers, of the radio frequency signal at the respective frequency corresponds to interrogating a respective predefined area at a predefined sweep frequency. The respective predefined area and predefined sweep frequency is modified based at least in part on the reflected radio frequency signals and frequency-shifted re-transmitted signals.

According to one or more embodiments, an orientation of the active radio frequency beacon in the three-dimensional space can determined based at least in part on accelerometer data and/or location information associated with two or more antennae.

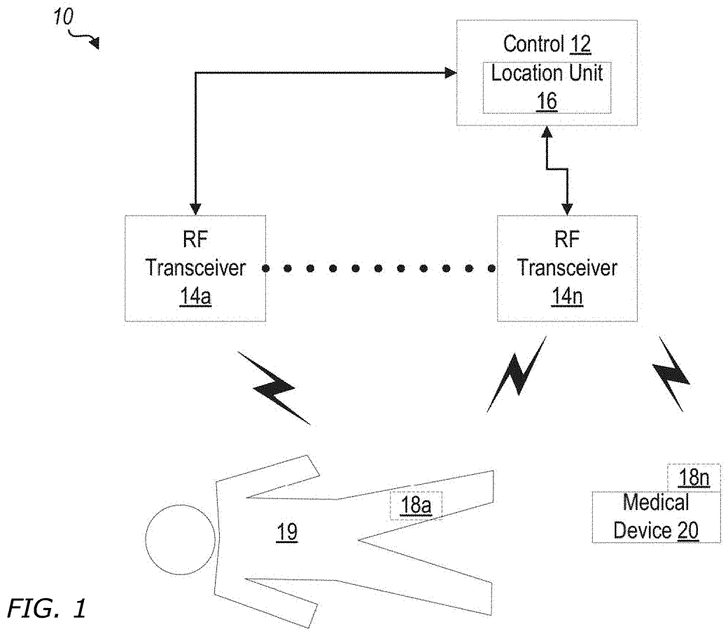

Referring now to the drawing figures, in which like elements are referred to by like reference numerals, there is shown in FIG. 1 a schematic diagram of a system 10, which comprises control device 12 in communication with radio frequency (RF) transceiver 14a-14n (collectively referred to as RF transceiver 14). Control device 12 may include location unit 16 for performing one or more control device 12 functions as described herein such as with respect to object location in a three-dimensional space. System 10 further includes active RF beacons 18a-18n (collectively referred to as active RF beacon(s) 18) that are configured to communicate one or more signals in response to an interrogation signal from RF transceiver 14 in a medical environment, for example, as described herein. Active RF beacon 18 may be removably attached to a device or other medical object, e.g., a pin may be used to attach it to a person 19 or patient 19. In one or more embodiments, active RF beacon 18 is removably attached/attachable to a medical object such as medical device 20.

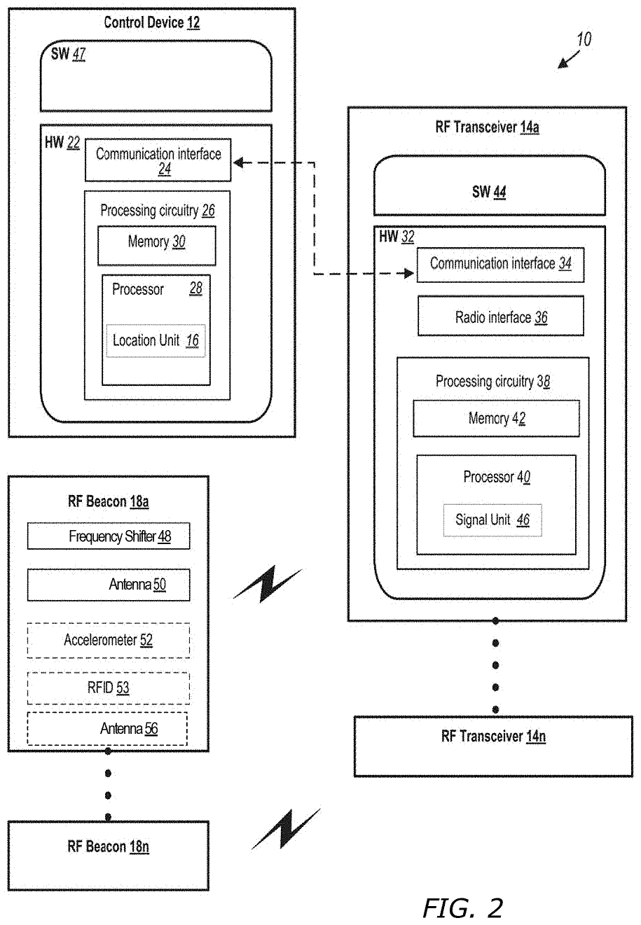

FIG. 2 is a block diagram of an example system 10 according to one or more embodiments of the invention. The system 10 includes a control device 12 that includes hardware 22 enabling it to communicate with RF transceivers 14. The hardware 22 may include a communication interface 24 for setting up and maintaining a wired or wireless connection with an interface of a different device such as RF transceiver 14 of the system 10.

In the embodiment shown, the hardware 22 of the control device 12 further includes processing circuitry 26. The processing circuitry 26 may include a processor 28 and a memory 30. In some embodiments, in addition to or instead of a processor, such as a central processing unit, and memory, the processing circuitry 26 may comprise integrated circuitry for processing and/or control, e.g., one or more processors and/or processor cores and/or FPGAs (Field Programmable Gate Array) and/or ASICs (Application Specific Integrated Circuitry) adapted to execute instructions. The processor 28 may be configured to access (e.g., write to and/or read from) the memory 30, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory). Memory 31, allowing additional storage capability, such as for instructions or data associated with received RF signals, can also be accessed by control device 12 and processing circuitry 26, including processor 28 and location unit 16.

As shown in the example of FIG. 2, the control device 12 further has software stored internally in, for example, memory 30, memory 31, or stored in external memory (e.g., database, storage array, network storage device, etc.) accessible by the control device 12 via an external connection. The software 47 may be executable by the processing circuitry 26. The processing circuitry 26 may be configured to control any of the methods and/or processes described herein and/or to cause such methods, and/or processes to be performed, e.g., by control device 12. Processor 28 corresponds to one or more processors 28 for performing control device 12 functions described herein. Memory 30 and memory 31 are configured to store data, programmatic software code and/or other information described herein. In some embodiments, the software 47 may include instructions that, when executed by the processor 28 and/or processing circuitry 26, causes the processor 28 and/or processing circuitry 26 to perform the processes described herein with respect to control device 12. In some embodiments, processing circuitry 26 of the control device 12 may include location unit 16 configured to perform one or more control device 12 functions as described herein such as with respect to active RF beacon location.

The system 10 further includes an RF transceiver 14 that includes hardware 32 enabling it to communicate with the control device 12 and/or active RF beacon 18. The hardware 32 may include a communication interface 34 for setting up and maintaining a wired or wireless connection with an interface of different devices of the system 10 such as control device 12, as well as a radio interface 36 for wirelessly communicating with RF beacon 18, as described herein. The radio interface 36 may be formed as or may include, for example, one or more RF transmitters, one or more RF receivers, and/or one or more RF transceivers.

In the embodiment shown, the hardware 32 of the RF transceiver 14a further includes processing circuitry 38. The processing circuitry 38 may include a processor 40 and a memory 42. In some embodiments, in addition to or instead of a processor, such as a central processing unit, and memory, the processing circuitry 38 may comprise integrated circuitry for processing and/or control, e.g., one or more processors and/or processor cores and/or FPGAs (Field Programmable Gate Array) and/or ASICs (Application Specific Integrated Circuitry) adapted to execute instructions. The processor 40 may be configured to access (e.g., write to and/or read from) the memory 42, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory).

As shown in the example of FIG. 2, the RF transceiver further has software 44 stored internally in, for example, memory 42, or stored in external memory (e.g., database, storage array, network storage device, etc.) accessible by the RF transceiver 14 via an external connection. The software 44 may be executable by the processing circuitry 38. The processing circuitry 38 may be configured to control any of the methods and/or processes described herein and/or to cause such methods, and/or processes to be performed, e.g., by RF transceiver. Processor 40 corresponds to one or more processors 40 for performing RF transceiver 14 functions described herein. The memory 42 is configured to store data, programmatic software code and/or other information described herein. In some embodiments, the software 44 may include instructions that, when executed by the processor 40 and/or processing circuitry 38, causes the processor 40 and/or processing circuitry 38 to perform the processes described herein with respect to RF transceiver 14. In some embodiments, processing circuitry 38 of the RF transceiver 14 may include a signal unit 46 configured to perform one or more RF transceivers 14 functions described herein such as with respect to transmitting and/or receiving wireless signals.

System 10 includes one or more active RF beacons 18 where each active RF beacon 18 may include a frequency shifter 48, antenna 50 (which can be a conical or horn component in some implementations), an optional accelerometer 52, and an optional second antenna 56. In particular, the RF transceivers 14 and control device 12 can be configured to track the active RF beacon 18, which may be removably attached to the exposed surface of a bone (in which case, attaching the RF beacon 18 does not require separate incisions). The active RF beacon 18 is configured to emit re-transmitted, frequency shifted RF signals (such as using frequency shifter emitter 48) to generate pseudo Doppler shifts. This frequency-shifted signal may provide additional interference waves to indicate the RF beacon 18's location down to sub millimeter accuracy, e.g., 1 millimeter accuracy with an error of less than 1 millimeter.

In one or more embodiments, a motor in a device such as surgical saw or drill may provide vibrations that, for particular frequency-shifted values though frequency shifter 48— where the pseudo Doppler shifts from the frequency shifter 48 interfere with receding and approaching surfaces of the motor blade—have the ability to average the determined location based on the re-transmitted RF signal to a point (or approximately a point) and that improves location determination of the vibrating device in combination with the active RF beacon 18.

Accelerometer(s) 52 may be used to detect and monitor movement and/or orientation of the active RF beacon 18 and provide instantaneous feedback of the X, Y and Z coordinates to control device 12 for real-time tracking. For example, in a single accelerometer/gyro 52 combination, pitch roll yaw can be determined for orienting RF beacon 18 in 3D space. The data from accelerometer 52 may be transmitted to control device 12 via one or more wireless communication protocols via a radio interface of active RF beacon 18 and the control device 12 can use the data to determine a point location of active RF beacon 18 and accelerometer orientation for plane. The plane may define the bone or other medical object orientation with respect to RF transceivers 14. The wireless communication protocols may include BLUETOOTH.

In one or more embodiments, antenna 50 (which can be a conical or horn component) can be configured to re-transmit RF signals from one or more RF transceivers 14, in an efficient path back to one or more RF transceivers 14. In some embodiments, the antenna 50 may be a device that spins to reflect the RF signals from RF transceiver 14. In one or more embodiments, the spinning of the antenna 50 may be triggered by receiving the RF signal and/or it may spin periodically or continuously while powered. In one or more embodiments, active RF beacon 18 may include a radio frequency identification (RFID) 53 that may be embedded on the reflected signal and/or RFID 53 may generate a separate RF signal indicating the RFID. In embodiments, active RF beacon 18 can include a second antenna 56 which can emit a further modified RF signal. In an embodiment, control device 12 can be configured to separately determine the locations of antenna 50 and antenna 56 which, together, can provide information about the orientation of active RF beacon 18.

In one or more embodiments, the one or more frequencies used herein may be modified to keep the RF beacons 18 within a predefined band. The system 10 may be calibrated with other frequency generators such as a saw or drill at least in part by determining the unique signal signatures for these devices or frequency generators. The software described herein may filter these frequencies and assign unique frequencies to the beacons to prevent noise generation. Once the system uniquely identifies the active RF beacons 18, the location and/or position of the active RF beacons 18 may be used for determining final implant placement, for example.

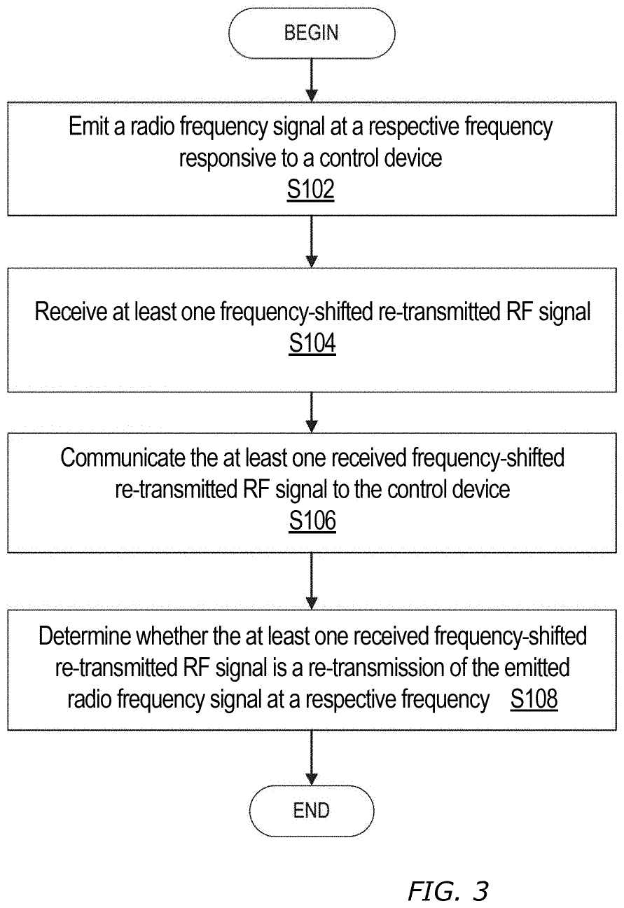

FIG. 3 is an example flowchart of a process implemented by RF transceiver 14 according to one or more embodiments of the invention in cooperation with control device 12. One or more Blocks and/or functions performed by RF transceiver 14 and control device 12 may be performed by one or more elements of RF transceiver 14 and control device 12, such as by signal unit 46, processing circuitry 38, processor 40, processor 28, etc. In one or more embodiments, RF transceiver 14, such as via one or more of signal unit 46, processing circuitry 38, processor 40, radio interface 36, etc. is configured to emit (Block S102) a radio frequency signal at a respective frequency, as described herein. For example, RF transceiver 14 can emit an RF signal responsive to transmission instructions issued by control device 12. In one or more embodiments, RF transceiver 14, such as via one or more of signal unit 46, processing circuitry 38, processor 40, radio interface 36, etc., is configured to receive (Block S104) at least one re-transmitted frequency-shifted RF signal, as described herein.

In one or more embodiments, RF transceiver 14, such as via one or more of signal unit 46, processing circuitry 38, processor 40, radio interface 36, etc., is configured to communicate data relating to the at least one received frequency-shifted re-transmitted RF signal to the control device 12 (Block S106), as described herein. In one or more embodiments, RF transceiver 14 in cooperation with control device 12, such as via one or more of signal unit 46, processing circuitry 38, processor 40, processor 28, etc., is configured to determine whether the at least one received frequency-shifted re-transmitted RF signal is a re-transmission of the emitted radio frequency signal at the respective frequency (Block S108), as described herein. Such a determination may be made according to timing, and/or may be made according to the uniqueness or distinctiveness of a frequency-shift associated with a particular active RF beacon 18 and/or associated with one antenna (50 or 56) on one particular active beacon 18. For example, each RF transceiver 14 in a constellation may emit RF signals or pulses in a timed sequence, thereby ensuring that re-transmitted signals (from all active RF beacons) will return to the RF transceiver 14 that emitted the RF signal before a different RF transceiver 14 sends out its own RF signal pulse.

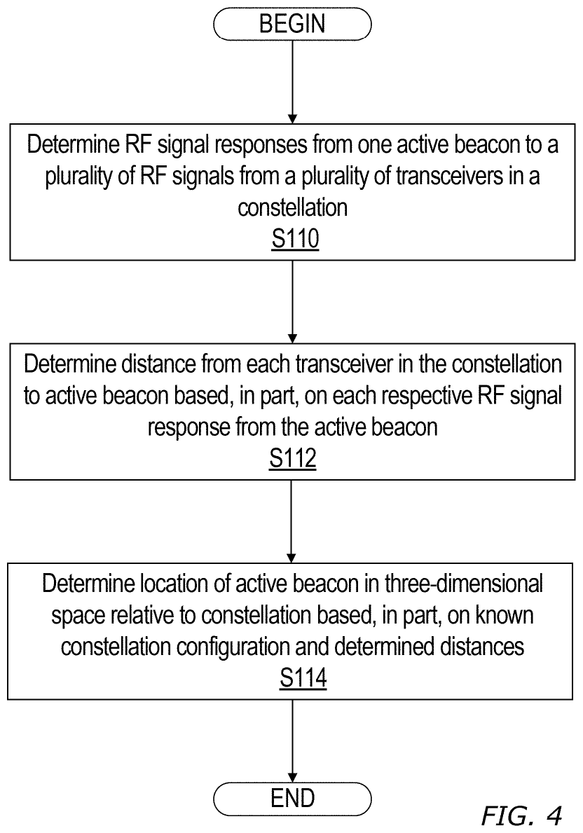

FIG. 4 is an example flowchart of a process of control device 12 according to one or more embodiments of the invention. One or more Blocks and/or functions performed by control device 12 may be performed by one or more elements of control device 12, such as by location unit 16, processing circuitry 26, processor 28, etc. In one or more embodiments, control device 12, such as via one or more of location unit 16, processing circuitry 26, processor 28, etc., is configured to determine or identify (Block S110) the plurality of RF signal responses that are associated with one particular active RF beacon 18 that was responding to RF signals from the plurality of transceivers in a constellation. In some embodiments, such a determination can be made according to a method consistent with FIG. 3.

As noted with respect to FIGS. 1 and 2, some embodiments of the system 10 includes a plurality of radio frequency transceivers 14a-n arranged in a constellation, as described herein. Each of the plurality of radio frequency transceivers 14 in the constellation (e.g., transceiver 14a, transceiver 14b, transceiver 14c) are configured to emit a radio frequency signal at a respective frequency (e.g., at a frequency that is distinct or unique relative to the frequencies used by the other radio frequency transceivers in the system 10) into an area that includes active RF beacons 18a-n. Similarly, the plurality of radio frequency transceivers 14a-n are configured to receive frequency-shifted RF signals from the surgery area, i.e., the area of interest, where the frequency-shifted RF signals originate from one or more of the active beacons 18a-n.

As noted previously, in some embodiments, each radio frequency beacon 18, e.g., RF beacon 18a, may be removably attachable to a medical object (e.g., to a bone or a medical tool), and the radio frequency beacon 18a may be configured to re-transmit or emit a frequency-shifted RF signal in response to receiving a different respective radio frequency signal from each of the plurality of radio frequency transceivers 14a-n. In some embodiments, the frequency-shifted RF signal may be produced using the frequency-shifter 48. In one or more embodiments, the re-transmitted frequency-shifted radio frequency signals are received or detected by one or more of the radio frequency transceivers 14a-n in the constellation. In various embodiments, the radio frequency transceivers 14a-n communicate data describing or representing the received frequency-shifted radio frequency signals to the control device 12, which processes the data as described herein.

Referring again to FIG. 4, in various embodiments, the processing circuitry 26 of the control device 12 may be configured to determine or calculate a distance (Block S112) from each transceiver 18a-n in the constellation to the active RF beacon 18a. For example, based on the time delay between the emission of the RF signal from the RF transceiver 14a and the reception of the re-transmitted frequency-shifted signal from the active beacon 18a, a distance to the active beacon 18a can be calculated or determined using the well-known speed of RF signals.

In some embodiments, an active RF beacon 18a can be configured with a reflector component, (which can be a conical component, a horn, and/or also function as antenna 50), which can be used by the system 10 to calibrate any inherent, constant, time delay that is associated with active beacon 18a re-transmitting a frequency-shifted signal. For example, RF transceiver 14a configured to function as conventional radar can observe, receive, or detect a reflected RF signal from active beacon 18a, as well as the re-transmitted frequency-shifted signal, and the system 10 can use the data describing or representing the reflected RF signal to calculate or determine the time delay added by the active RF beacon 18a before it emits a re-transmitted frequency-shifted signal back to the RF transceiver 14a. In some embodiments, such a time delay may be associated with the time it takes for the RF beacon 18a to process the incoming, received RF signal and to produce the outgoing frequency-shifted RF signal that it emits. Control device 12 can be configured to use this time-delay calibration information in determining the distance to active beacon 18a based on the re-transmitted frequency-shifted signal.

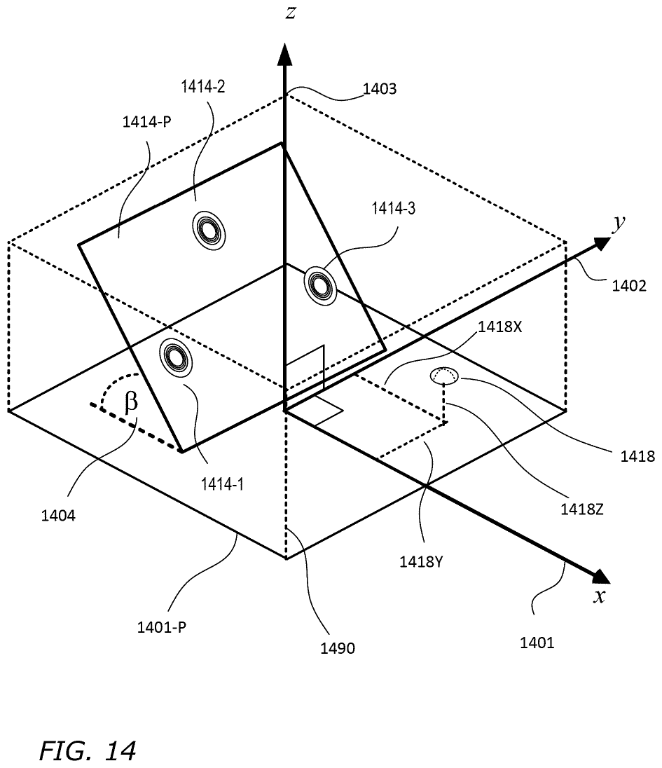

Based upon the determined distances from each RF transceiver 14a-n to a particular active RF beacon 18a and upon the known configuration of the constellation of RF transceiver 14a-n, the control device 12 can determine the location of active beacon 18a in three-dimensional space (Block S114), e.g., using trilateration or similar techniques known in the art. This is described further in FIG. 14 below.

Referring again to FIGS. 1 and 2, additional embodiments are described in the following paragraphs. According to one or more embodiments, the radio frequency beacon 18a includes an antenna 50 (which may be conical, and may provide some reflection of RF signals) where the antenna 50 is configured to re-transmit radio frequency signals. According to one or more embodiments, the plurality of radio frequency transceivers 14 are configured to interrogate a respective predefined area at a predefined sweep frequency where the control device 12 is configured to modify the respective predefined area and predefined sweep frequency based at least in part on the location of the medical object.

According to one or more embodiments, the medical object is one of a surface of a bone and a medical device. According to one or more embodiments, the determination of the location of the medical object in three-dimensional space includes determining, for each respective reflected radio frequency signal, a respective location in three-dimensional space of the medical object. The determined location of the medical object in three-dimensional space is based on the determined respective locations in three-dimensional space of the medical object.

According to one or more embodiments, the active radio frequency beacon 18a includes at least one accelerometer 52 configured to generate accelerometer data where at least one of the reflected radio frequency signals including the accelerometer data. According to one or more embodiments, the control device 12 is further configured to determine an orientation of the radio frequency beacon 18 in the three-dimensional space based at least in part on the accelerometer data.

In one or more embodiments, pulsed waves, i.e., RF signals, at various frequencies, for example, 3 to 300 GHz, are transmitted by RF transceiver 14 such as via radio interface 36 to track the distance and speed of an object based on the return of the signal and its modified frequency. Such changes in frequency response can be identified, characterized, and classified as unique signals such as by RF transceiver 14a and/or control device 12. In one or more embodiments, RF transceivers 14a-n can be used to triangulate (and/or trilaterate) the location of an RF beacon 18a that returns or provides RF waves more efficiently than the surrounding objects. In one or more embodiments, the RF transceivers 14a-n may triangulate (or trilaterate) the location of the active RF beacon 18a based at least in part on the frequency-shifted signals from frequency shifter 48 of RF beacon 18a, where the results of the object triangulation (or trilateration) for the various signals (e.g., re-transmitted and frequency-shifted radio frequency signals) can be combined or processed with other localization and/or sensor data.

In other configurations, varying the frequency of the waves emitted by the RF transceivers 14a-n can allow the locating and positioning of the object to be more accurate. If the object's positional accuracy is required to be submillimeter, waves of multiple wavelengths may be used by control device 12 to determine the location and avoid misjudging the location of an object whose location is in between the wavelengths.

Having generally described arrangements for RF beacon 18a location monitoring, examples of details for these arrangements, functions and processes are provided as follows, and various of these arrangements, functions and processes may be implemented by the control device 12 and/or RF transceiver 14 in some embodiments.

Object Triangulation or Trilateration:

In one or more embodiments, signals radiated from an RF transceiver 14a may be scattered from any material in the operating room, i.e., predefined area/environment, including from the personnel performing the surgery. These scattered signals can be filtered out by looking at Doppler offsets since each of the active RF beacons 18a-n may be configured to return specific frequency-shifted signals (or re-radiate signals at known “Doppler” offsets). In one or more embodiments, Doppler filtering is configured to allow for the detection of weak signals in the presence of strong clutter by, at least in part, differentiating moving object signatures from static object signatures. An object signature may correspond to one or more RF signal transmitted and/or reflected (or re-transmitted) by an object.

In one or more embodiments, three RF transceivers 14a-c are located around the region of interest which contains an RF beacon 18a, which may be configured, for example, for bone tracking for orthopedic applications, or for tool tracking, for example tracking a bone saw or drill for surgical applications. In one or more embodiments, the three RF transceivers 14a-c emit waves at varying frequencies in cascading pulses of milliseconds, therefore each returning wave to the RF transceivers 14a-c may be from a different frequency.

In one or more embodiments, the three RF transceivers 14a-c may be in a circular configuration, for example located on an OR light handle, and a fourth RF transceiver 14d for better triangulation or trilateration of the active RF beacons 18 may also be used. In some embodiments, an active RF beacon 18a may be removably attached to a bone or the like using pins. A method for tracking active RF beacons 18a using all three RF transceivers 14a-c, fixed with respect to each other in a circular arc with 120 degrees of separation between the RF transceivers may be used.

In some embodiments, control device 12 may be used to determine how the submillimeter differences in the location of the radar transceivers affect each change in distance. In one or more embodiments, a laser range finder may be attached to the RF transceivers 14a-n to determine true distance from the radars prior to the surgery (referred to herein as preoperative, or preop calibration). Once the ranges are set, wavelengths of appropriate frequency for each RF transceiver 14 may be used for that range of distance to yield the readings for the tools and bones, which may help improve accuracy of the distance determination.

In one or more embodiments, RF transceivers 14a-n may trace their available field of vision with an arrayed approach with fixed vision. This means that the RF transceivers 14a-n, such as via one or more of processing circuitry 38, signal unit 46, radio interface 36, etc., can sweep the area at a high frequency with constructive and destructive waves that couple. Once the RF transceivers 14a-n detect the returned waves or re-transmitted waves such as from active RF beacon 18a, the RF transceivers 14a-n may “lock” in on this region of interest (ROI) and sweep this area at a higher frequency, i.e., processing circuitry 38 reduces the field of vision for frequency sweeping. If the object associated with the active RF beacon 18a moves out of this area as may be determined by processing circuitry 38 due to a lack of a detected return signal, the RF transceiver 14a may re-sweep the available field of vision to find the active RF beacon 18a and corresponding object, and provide feedback to control device 12 if the object associated with an active RF beacon 18a is not found. Further, in one or more embodiments, laser range finders can be utilized to improve the accuracy of the distance determined from radar; e.g., to improve radar wavelength determination. Further, while system 10 is often described as using three RF transceivers 14a-c, the teachings herein are equally applicable to other quantities of RF transceivers such as less than 3 and/or greater than 3.

### Example Technique for Using Objection Location

After exposure for performing knee arthroplasty (total or partial), prior to scanning the bone, two screws may be placed in each bone, one in the distal femur and one over the proximal tibia. In some embodiments, the pin or screw is hollow and can accept an active RF beacon 18. Each active RF beacon 18 may have an RFID device 53 and a resonating feature and may have a QR code printed on the surface. This QR code can be customized based on the patient's anatomy, choice of implant and surgeon's preference prior to surgery.

In some embodiments, a 3D laser scanner may be used during surgery to scan the bony and cartilage surface, including the active RF beacon 18. In some embodiments, Radio Frequency Identifiers (RFID) are used to determine the unique part number of each pin and differentiate the pins in surgery. The code from the RFID is recognized by the RF transceiver 14 and/or control device 12 and the pre-operative loaded library of joint images, preferences and implant sizes are loaded.

The scan may then be uploaded to a cloud-based platform that is accessible by at least control device 12. The data are analyzed by, for example, an AI/ML algorithm based on an automated script that identifies landmarks for the featured bone and bony/soft-tissue landmarks are identified. This scan may then be superimposed on pre-operative images, if available, for a better registration process. A masking feature may be used to train the script to identify and better overlay the point clouds to each other with an RMS error minimizing algorithm.

While the scan is being analyzed, the patient's joint may be put through range of motion, for the example of a knee, flexion and extension of the knee is assessed. Then the knee is subjected to testing, through manual varus/valgus tests, to assess the soft tissue. The two active RF beacons 18a-b can be tracked during this process by system 10 and the change in the distance is analyzed, such as by control device 12 via processing circuitry 26 and/or location unit 16, as a change in the gaps during knee range of motion.

A cutting tool (e.g., medical object), such as a bone saw or a cutting block that helps the surgeon make the cuts, can be tracked during surgery using a third active RF beacon 18c and can be placed in the appropriate location to achieve the planned surgery. Cutting devices may also have an active RF beacon 18 and/or RF transceiver 14 attached to them to track and find landmarks that identify the location of cut planes or bone interaction locations to modify the surface.

In various embodiments, machine learning algorithms executed, for example, by control device 12 and/or RF transceiver 14, are used to assess the optimum position of the implant based on prior patient outcomes. For example, patient types are clustered to individual specialized groups based on multiple parameters using regression analysis, such as via processing circuitry 26. Control device 12 may identify the patient and find the best outcomes from previous surgeries performed on this patient type to prescribe the best cutting planes to replicate the best outcomes. Parameters of the implant alignment can be set pre-operatively to expedite this process.

In some alternative embodiments, a 3D scanner can be mounted over the cutting tool, such as an oscillating saw or drill. The scanner can detect the already scanned surface through object recognition software and demonstrate the proposed cutting/drilling planes that are to be executed.

In some alternative embodiments, a universal cutting jig is used that accommodates the tracking pin, i.e., the pin 72 used with an RF beacon 18, as a fixed point. A manual jig that is tracked by the RF transceivers 14 and that has a flat surface is positioned over the cutting block. The cutting block is now being tracked as compared to other tracking pins, i.e., with RF beacon 18, for both the femur and the tibia, separately, and pinned in place. The accurate position of the cutting block is shown on the monitor.

In some alternative embodiments, augmented reality while the surgeon is wearing a headset in communication with control device 12 is used to assess the accurate positioning of the cutting block or the cutting plane of the saw.

In some alternative embodiments, a robotic cutting tool can be used to execute the bony cuts. After the cuts are made and trial implants are placed, the knee is put through its range of motion and stressed to assess soft-tissue tension and post-cut kinematic data. In some embodiments, artificial intelligence implemented by control device 12, for example, is used to determine the landmarks and detect the axes of the bone based on prior cases.

In various embodiments, the combination of artificial intelligence and machine learning software, which may be executed in the cloud and/or control device 12, may eliminate the typically required advanced pre-operative imaging such as MRI or CT over time. X-rays can be used in adjunct to the intra-operative scan to determine the bone alignments.

In various embodiments, the 3D scan and radar coordinates are relayed and stored in the cloud computing service in communication with control device 12 and/or stored at control device 12. The coordinates may be converted into data to train machine learning algorithms, which are then used to build a mathematical model of training data. Every surgery builds a library of data and algorithms. These datasets may be continuously fed into the machine learning platform that may then cycle back to each, as described herein, for purposes such as identifying bony surfaces and generating cutting planes, which may be tailored to the patient's unique soft tissue balance and alignment, as well as the surgeon's preference. The RF transceivers 14a-n can also be used to make measurements after the cuts to determine the accuracy of the cuts to report back to the surgeon and/or to conduct validation.

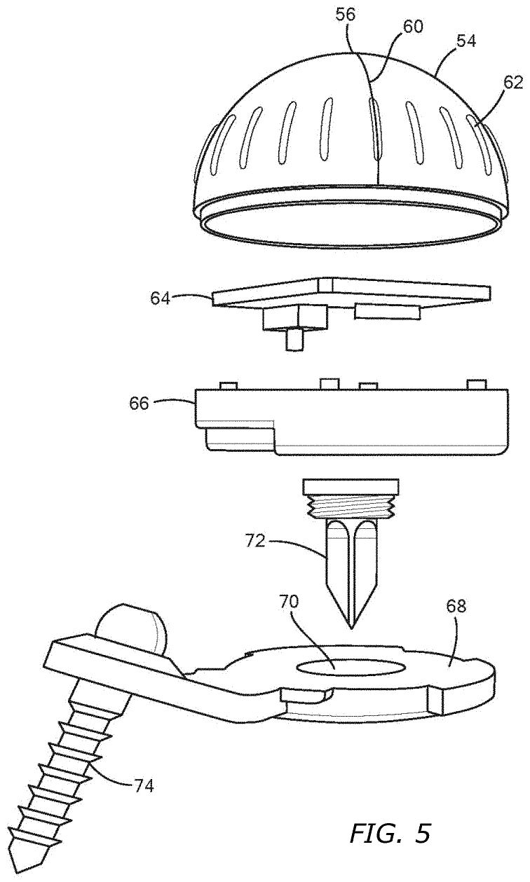

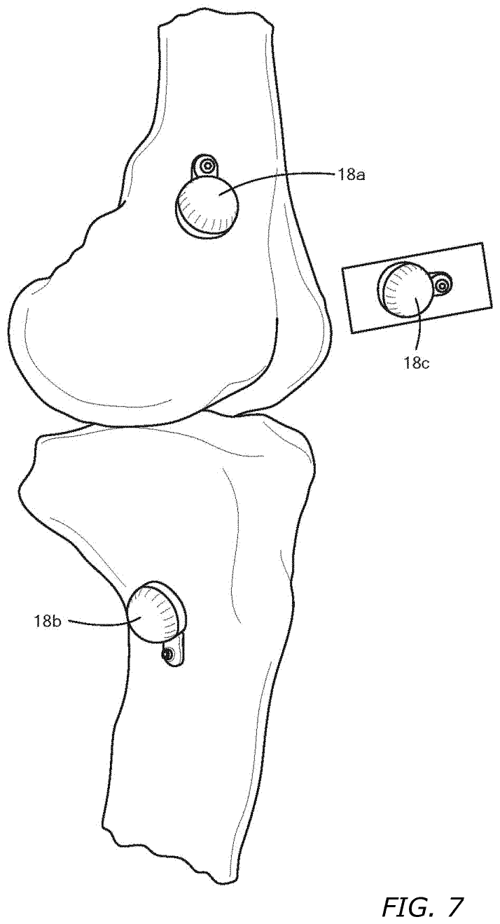

Referring now to FIGS. 5-7, active RF beacons 18a-n may be sized and configured to be releasably attached to a medical object, for example, a bone (shown in FIG. 7) or a cutting instrument of a robotic arm. For example, active RF beacon 18a may be anchored to the distal end of the femur, active RF beacon 18b may be anchored to the proximal end of the tibia, and active RF beacon 18c may be anchored to the cutting instrument of the robotic arm. Each active RF beacon 18 may include a dome 54 which, in one configuration, can have a diameter in a range, for example, from approximately 0.5 cm to approximately 3.0 cm. For example, in one configuration, each active RF beacon 18 may include a dome 54 which can have an approximate diameter selected from one of: 0.5 cm, 0.6 cm, 0.7 cm, 0.8 cm, 0.9 cm, 1.0 cm, 1.1 cm, 1.2 cm, 1.3 cm, 1.4 cm, 1.5 cm, 1.6 cm, 1.7 cm, 1.8 cm, 1.9 cm, 2.0 cm, 2.1 cm, 2.2 cm, 2.3 cm, 2.4 cm, 2.5 cm, 2.6 cm, 2.7 cm, 2.8 cm, 2.9 cm, or 3.0 cm. The dome 54 can include an antenna 50 and/or 56 disposed therein and indicator line 60 may extend from the base of the dome 54 to the apex. A plurality of gripping elements 62 may be disposed around the circumference of the dome 54 to provide tactile feedback to the physician when the dome 54 is touched. Subjacent to the dome may be a circuit board 64, for example, a PCB which includes the electronics of the active beacon 18. The circuit board 64 may include circuitry configured to cause a Doppler shift in the received RF signal. For one example, the circuitry is configured to actively modify the incoming first RF frequency and shift the frequency to a second RF frequency different than the first frequency as discussed herein. The frequency shift for each beacon 18 can be programmed such that each beacon 18 can shift the incoming frequency by a predetermined amount, which amount is preferably distinguishable from the predetermined amounts of frequency shift used by the other beacons 18. Coupled to the circuit board 64 may be an antenna extending upward into the dome 54 and an accelerometer in some embodiments.

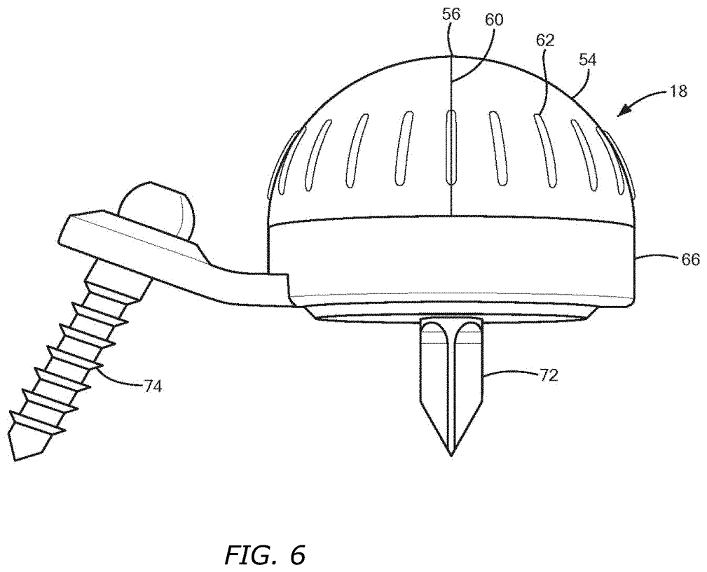

Continuing to refer to FIGS. 5-7, the circuit board 64 is sized to be received or otherwise coupled to a housing 66 which is coupled to the dome 54. In an exemplary configuration, the housing 66 defines a diameter commensurate in size with the maximum diameter of the dome. As shown in FIG. 6, the dome 54 is sized to couple with the housing and together with the housing to retain the circuit board 64 therein. Subjacent to housing 66 is a platform 68 sized and configured to releasably mount the dome 54 and the housing 66. In an exemplary configuration, the housing 66 is configured to twist-lock with the platform 68, which may further align the apex of the dome 54 to be parallel with the axis of the platform 68. The platform 68 may further define an aperture 70 therethrough in which a first fixation element 72 may be disposed and extend orthogonally from the platform 68. The first fixation element 72 includes a plurality of threads to releasably attach to the platform 68 and may define a cross-shape extending from the threads to aid in the initial purchase of the bone. In particular, the cross shaped design facilitates initial rotational stability and penetration on the cortex of the bone. Extending at an oblique angle from the platform 68 and spaced a distance from the aperture is a second fixation element 74. In the configuration shown in FIGS. 6 and 7, the platform 68 has a tilt that accommodates and is designed for the curvature of the distal medial femur and the proximal tibia. The second fixation element 74 facilitates overall stability of the platform 68.

In another embodiment, a wireless, radio frequency (RF) communicating device (e.g. Bluetooth, wifi) is utilized to achieve a six degree of freedom (DOF) tracking system where position and orientation of tracking is provided by one or more of Inertial Measurement Unit (IMU) sensor (such as an accelerometer, a gyroscope, a magnetometer, or the like). A secondary positional tracking source using RF signals can establish three DOF positions. Using the 3 DOF radar data will achieve the correct interpolation noise or drift errors from IMU based tracking. The secondary system can operate synchronously or asynchronously from a primary IMU based tracking system.

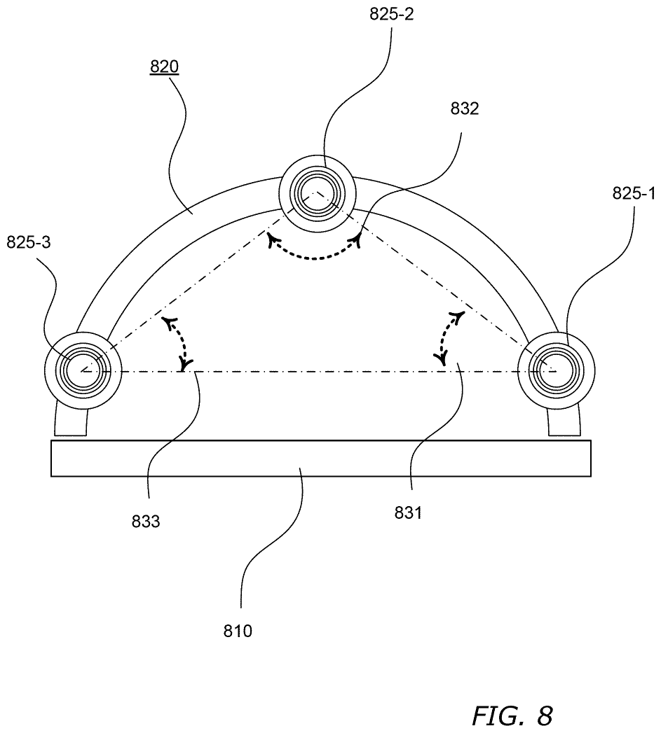

FIGS. 8-10 depict further embodiments consistent with this disclosure. In FIG. 8, a support 820 is depicted over an operating bed 810 or the like. Affixed to support 820 in a constellation configuration are a first RF transceiver 825-1, a second RF transceiver 825-2, and a third RF transceiver 825-3. In various embodiments, the RF transceivers 825-1, 825-2, and 825-3 may be the same as or similar to the RF transceivers 14a-14n described previously. In various embodiments, the RF transceivers 825-1, 825-2, and 825-3 each transmit at a different respective frequency in the range of approximately 60-66 GHz and each of the overlapping signals are unique frequencies or signals

In one embodiment, as shown, support 820 has an “arc” shape (e.g., the shape of a portion of a circle or of another curve) that is able to accommodate a patient 19 within or beneath the arc. Support 820 can include other elements (not shown in FIG. 8) associated with the embodiment, such as wiring, a power supply(ies), or control circuitry (e.g., such as control device 12 and/or hardware 32, among other things) for selecting a frequency range value, and/or for processing received, possibly modified as described herein, frequency values from a plurality of beacons (not shown in FIG. 8), in order to determine the location, in three-dimensional space, of each of the active radio frequency beacons as described herein (e.g., active RF beacon(s) 18). The support 820 may be made of any suitable material, such as plastic, fiberglass, resin, metal, or the like. In an embodiment, angles 831, 832, and 833 between the transceivers are fixed, and can be selected to provide optimum tracking information for each of the radio frequency beacons.

In one embodiment, multiple radar transceivers can be mounted in or affixed to a frame that encompasses a computer monitor/display. The shape of the radar frame can be similar to the outer shape of the monitor. Further still, the monitor and frame can be held on an articulating arm.

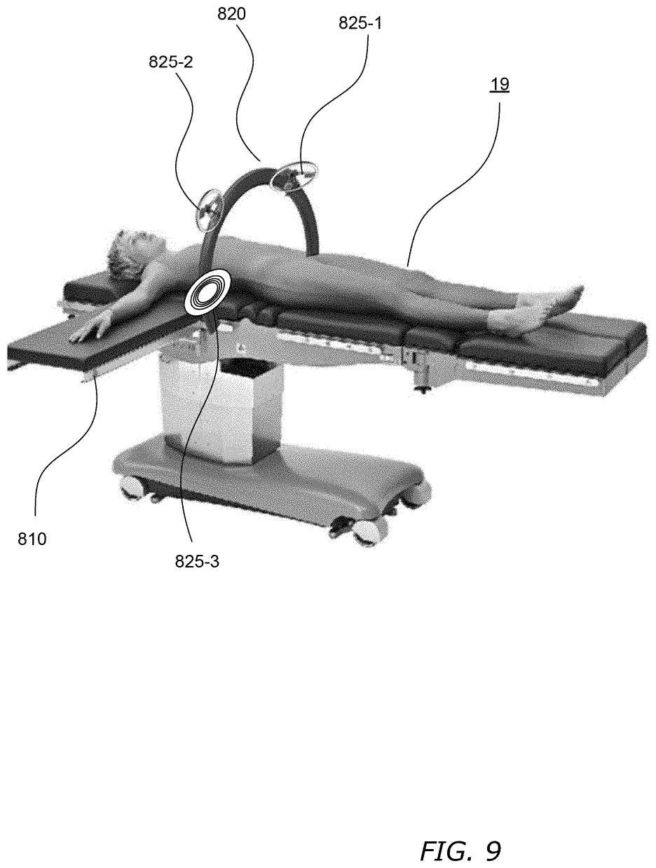

FIG. 9 shows a perspective view of an example of an embodiment of the support 820 and its constellation-configuration transceivers 825-1, 825-2, and 825-3 when affixed to the operating bed 810, upon which lies a patient 19 who is having surgery on their knee. In FIG. 9, there is no draping or other line-of-sight obstructions between the transceivers 825-1, 825-2, and 825-3 and the knee of the patient 19, which is where one or more of the radio frequency beacons 18 described herein will be attached during surgery.

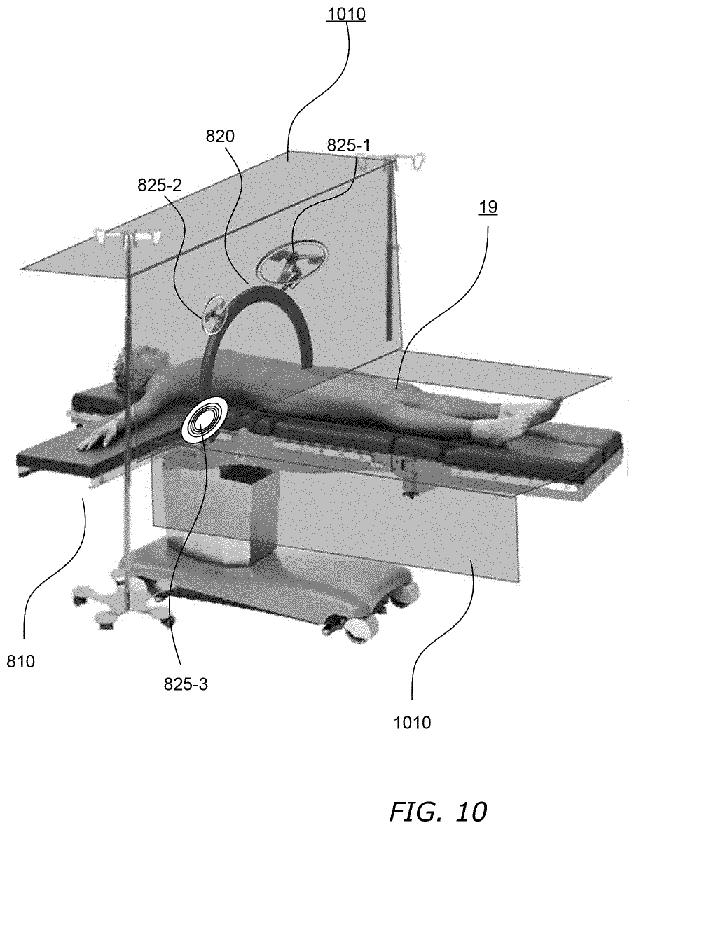

Consistent with this disclosure, at least one embodiment of a system for medical object tracking is able to operate in an environment where one or more part(s) of the system is obstructed, e.g., “draped,” for example as shown in FIG. 10, such that one or more of the radio frequency transceivers 825-1, 825-2, and 825-3 are on an opposite side of an obstruction, such as the sterile drape 1010, from the region of the operating environment that contains the medical objects that the system tracks. For example, location tracking consistent with this disclosure can occur in the region of a knee of the patient 19, as shown in FIG. 10, even though the line of sight between the knee 19 and the radio frequency transceivers 825-1, 825-2, and 825-3 are obstructed by the drape 1010. Consistent with this disclosure, the system functions in a draped environment because the sterile drapes 1010 are transparent or essentially transparent to the radio frequency signals used by the RF transceivers 825-1, 825-2, and 825-3 and/or by the active radio frequency beacons 18 described herein. In various embodiments described herein, the frequency(ies) of the RF signals transmitted by the RF transceivers 825-1, 825-2, and 825-3 and/or re-transmitted and frequency-shifted (or reflected) by the active RF beacons 18 may be chosen such that the material that forms the sterile drape 1010 does not substantially attenuate or otherwise alter the RF signals. In various embodiments, an obstruction (e.g., the sterile drape 1010) does not substantially attenuate the RF signals if the signal loss caused by the obstruction is approximately −6bB or less, such as −6 dB, −5 dB, −4 dB, −3 dB, −2 dB, or −1 dB. In such embodiments, the drape 1010 (or other obstruction) may be referred to as being transparent or essentially transparent to the RF signals at that frequency(ies).

As is known in the medical industry, a sterile drape 1010 is typically made of cloth, (such as a Polypropylene cotton fabric, a polyester fabric, a cotton-polyester blend fabric, or the like), plastic, or paper; all of which are transparent or essentially transparent to the radio frequency signals used by the embodiments described herein. In various other embodiments, the sterile drape 1010 may be made of any material that is transparent or essentially transparent to the radio frequency signals used by the embodiments described herein.

Although the embodiments shown in FIGS. 8-10 show examples where the support 820 has an arc or rectangular shape, in other embodiments, the support 820 may have a multisided shape that is arc-like or somewhat similar to an arc. For instance, the support 820 may be in the shape of a portion (e.g., a half) of a polygon, such as a hexagon, heptagon, octagon, decagon, dodecagon, or the like. Furthermore, although various embodiments described herein use a drape 1010 as an example of an obstruction, the system's operation and advantages apply also to other types of obstructions besides drapes, such as medical equipment, bedding, clothing, and other items that are present in an OR and that are made of a material that is transparent or essentially transparent to the RF signals described herein.

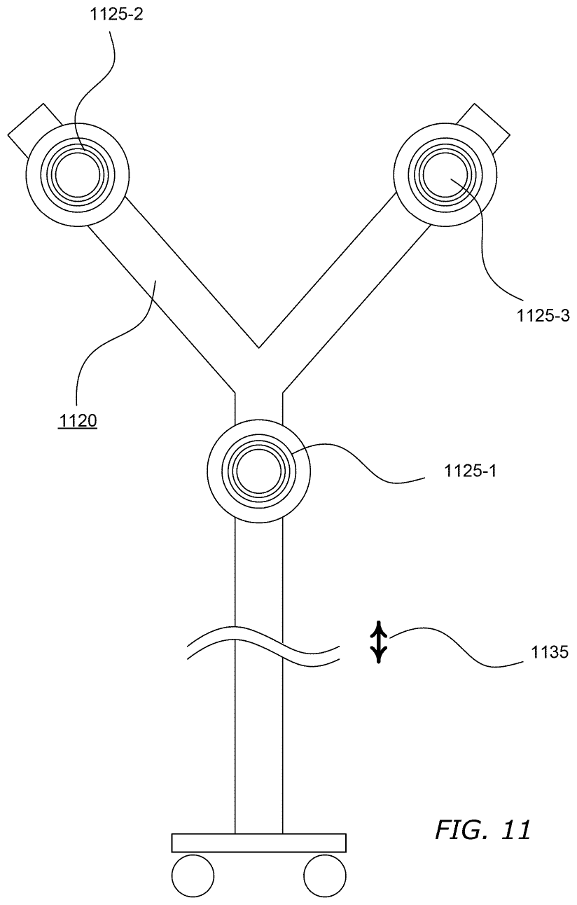

FIG. 11 depicts a further embodiment consistent with this disclosure. In FIG. 11, a support 1120 may be a Y-shaped stand that can be positioned near an operating environment where location tracking is desired; e.g., near the knee that is undergoing surgery for a patient 19. In some embodiments, as shown, the Y-shaped support stand 1120 may include wheels for easy movement and placement. In this example, affixed to support 1120 are a first transceiver 1125-1, a second transceiver 1125-2, and a third transceiver 1125-3 in a constellation configuration. As with FIG. 8, the first transceiver 1125-1, a second transceiver 1125-2, and a third transceiver 1125-3 may be the same as or similar to the RF transceivers 14a-14n described previously.

In one embodiment, support 1120 fixes the relative positions of first transceiver 1125-1, second transceiver 1125-2, and third transceiver 1125-3 to each other, but the overall height of the fixed transceiver constellation or array can be adjustable (as depicted by arrow 1135). Similar to what was described previously with respect to support 820, support 1120 can include other elements associated with the embodiment, such as control circuitry for selecting a frequency range value, and for processing received frequency values from a plurality of beacons (not shown in FIG. 11), in order to determine the location, in three-dimensional space, of each of the radio frequency beacons as described herein, (e.g., RF beacon(s) 18). As with the system depicted in FIGS. 8-10, the embodiment depicted in FIG. 11 can be draped without significantly degrading its operation despite the line of sight between the transceivers 1125 and the RF beacons being blocked by the drapes and/or other obstructions.

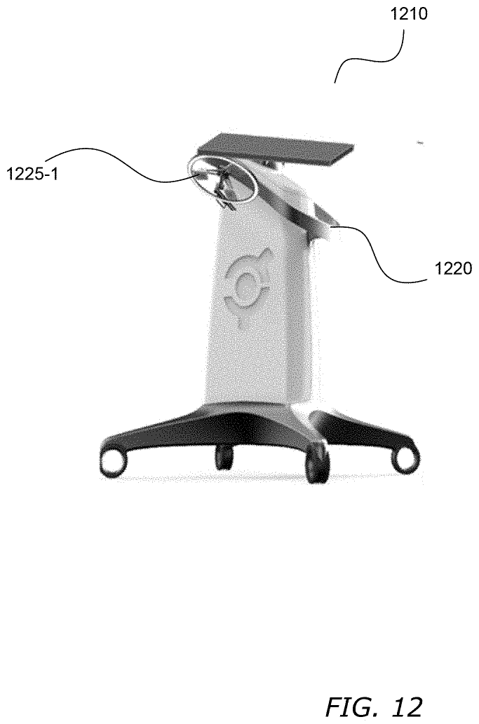

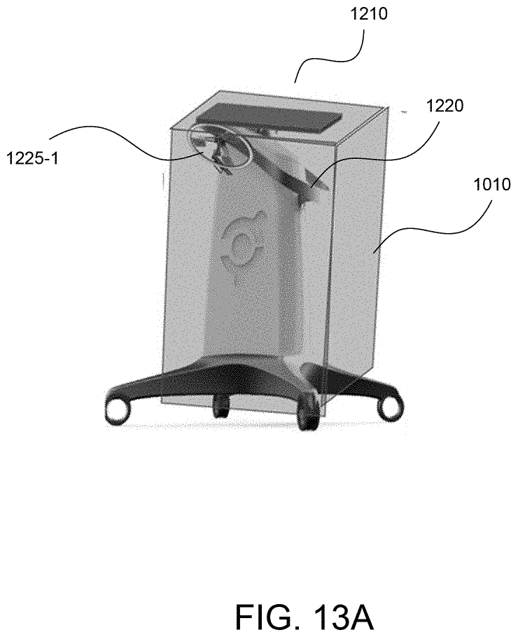

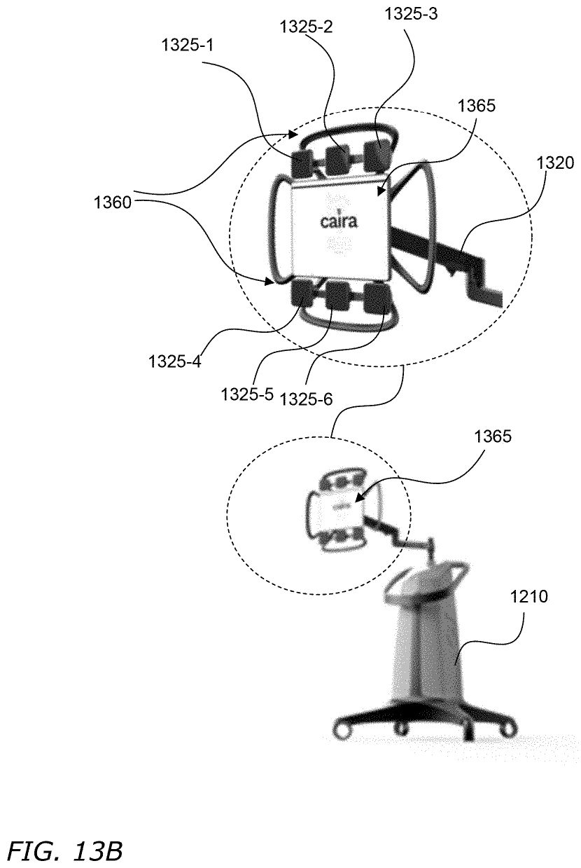

FIGS. 12, 13A, and 13B depict a further embodiment consistent with this disclosure. In FIG. 12, a stand 1210 can be or include a surgical table that can be moved around an operating environment. In some embodiments, as shown, the stand 1210 may include wheels for easy movement and placement. Affixed to stand 1210 is a support 1220, and fixed onto support 1220 are RF transceivers in a constellation configuration. (In the view of FIG. 12, only a first RF transceiver 1225-1 is shown.) In an embodiment, support 1220 fixes the relative positions of the RF transceivers 1225 in the constellation, and similar to what was described previously with respect to support 820, support 1220 and/or stand 1210 can include other elements associated with the embodiment, such as control circuitry for selecting a frequency range value, and for processing received, frequency values from a plurality of active RF beacons (not shown), in order to determine the location, in three-dimensional space, of each of the radio frequency active beacons as described herein, (e.g., active RF beacon(s) 18). As shown in FIG. 13A, the embodiment depicted in FIG. 12 can be draped or otherwise obstructed without significantly degrading its operation despite the line of sight between the transceivers 1125 and the RF beacons (not shown) being blocked by the drapes 1010 or another obstruction.

Further still, as discussed above, multiple radar transceivers can be mounted in or affixed to a frame that encompasses a computer monitor/display where the shape of the radar frame can be similar to the outer shape of the monitor, and the monitor and frame can be held on an articulating arm. Consistent with this disclosure, the articulating arm can be affixed to any appropriate object in the tracking environment. For example, an articulating arm may be affixed to an surgical table or bed, where a monitor may be in use. As shown in FIG. 13B, by way of example, rolling stand 1210 may support articulating arm 1320 which supports monitor 1365, where frame 1360 can be configured to encompass a plurality of transceivers 1325-1, 1325-2, 1325-3, 1325-4, 1325-5, and 1325-6 in a fixed configuration with respect to each other.

Further still, in one configuration, the absolute location and orientation of RF-transceivers 1414-1, 1414-2, and 1414-3 (and any additional fixed RF transceivers consistent with this disclosure) in a surgical operating environment can be registered and stored, for example, by the control device 12 in memory 31, thereby allowing the control device 12 to determine an absolute location and absolute orientation of any active RF Beacon(s) 18 with respect to the surgical environment. Such a determined absolute location value and absolute orientation value of active RF Beacon(s) 18 (at either a single time point, or as a function of time) can also be stored in memory 31. Where a representation of the surgical environment is provided on the computer monitor/display 1365, then the determination of the absolute location (and absolute orientation) of the RF Beacon(s) 18 also permits a representation of the active RF Beacon(s) 18, in the surgical environment, to be shown on the computer monitor/display 1365 consistent with this disclosure, either statically, or as a function of time.

FIG. 14 depicts an exemplary geometry for determining a location in three-dimensional space of active RF beacon 1418 relative to an exemplary constellation configuration associated with RF transceivers 1414-1, 1414-2, and 1414-3. In various embodiments, the first transceiver 1414-1, second transceiver 1414-2, and third transceiver 1414-3 may be the same as or similar to the RF transceivers 14a-14n described previously. By way of example only, during a surgical operation on a first patient, the three transceivers (RF transceivers 1414-1, 1414-2, and 1414-3) in a first constellation configuration can be configured to lie in a plane (1414-P) that, itself, exhibits a slant or skew (β angle 1404) relative to a plane (1401-P) defined by a surgical operating table or bed on which the first patient rests. (An exemplary surgical environment in FIG. 14 is depicted by the box-shaped region 1490.) Furthermore, the three transceivers (RF transceivers 1414-1, 1414-2, and 1414-3) can be configured to lie in an arc. One of ordinary skill in the art will appreciate that any number of “preset” constellation configurations (with varying skew angles, varying RF transceiver separations, and varying numbers of transceivers) can be accommodated consistent with this disclosure.

As disclosed herein, each of the transceivers (RF transceivers 1414-1, 1414-2, and 1414-3) can be configured to transmit RF signals at a particular, distinct RF frequency, (which is also referred to herein as a respective frequency for each of the transceivers). Techniques and systems disclosed herein can also include one or more active beacons (1418). As disclosed herein, an active beacon (1418) can be configured to be responsive to incoming RF signals from the plurality of transceivers (RF transceivers 1414-1, 1414-2, and 1414-3). Specifically, upon receipt of an incoming RF signal from one of the plurality of transceivers, an active beacon (1418) consistent with this disclosure can be configured to emit an RF signal that is modified relative to the RF signal it receives. More specifically, consistent with this disclosure, the modified RF signal emitted by an active beacon (i.e., the modified outgoing RF signal) can be a signal at a RF frequency that is shifted relative to the incoming RF signal received by the active beacon. For example, if the incoming RF signal has a 299 GHz frequency, then in response, the active beacon 1418 may shift it up (e.g., increase the frequency) by 5 GHz to 304 GHz and emit the 304 GHz RF signal back to the RF transceivers. Similarly, if the incoming RF signal has a 320 GHz frequency, then in response, the active beacon 1418 may shift it up by 5 GHz to 325 GHz and emit the 315 GHz RF signal back to the RF transceivers. As a further example, a second active beacon (not shown in FIG. 14) may shift the incoming RF signal down (e.g., decrease the frequency) by 5 GHz (e.g., from 299 GHz to 294 GHz and from 320 GHz to 315 GHz).

Accordingly, consistent with this disclosure, an active beacon (1418) disclosed herein can be configured to actively re-transmit the frequency-shifted RF signals upon receipt of the RF signals from a plurality of transceivers (RF transceivers 1414-1, 1414-2, and 1414-3). Each active beacon can be configured to re-transmit at a unique (or distinct) frequency-shifted value different from any other active beacon in the surgical environment.

Consistent with techniques and systems disclosed herein, a control device 12, (not shown in FIG. 14), in communication with the plurality of transceivers 1414-1, 1414-2, 1414-3, can be configured to provide transmission instructions to each of the plurality of transceivers 1414-1, 1414-2, 1414-3. Each of the transceivers 1414-1, 1414-2, 1414-3, in turn, can be configured to transmit an RF signal at a unique or distinct frequency into the surgical environment 1490, responsive to the transmission instructions from the control device 12. Moreover, because each of the transceivers 1414-1, 1414-2, 1414-3 can be configured to transmit at a unique RF frequency into the surgical environment 1490, and each of the active beacons can be configured to re-transmit a unique frequency-shifted RF signal back into the surgical environment 1490 based on the RF signal it receives, the control device 12 (together with each of the transceivers 1414-1, 1414-2, 1414-3 can be configured to recognize each re-transmitted signal from a single active beacon, where each re-transmitted signal is based upon one of the independently-emitted RF signals from the plurality of transceivers 1414-1, 1414-2, 1414-3 in the constellation. (See FIG. 3, for example.)

Based upon this information (i.e., the time of the original transmission from each of the transceivers, the time at which the re-transmitted frequency-shifted signal is received back at each of the transceivers, and the fixed location of each of the transceivers in the constellation relative to each other), the control device can be configured to determine a location of the active beacon (1418) relative to the constellation by trilateration or the like. For example, in a system consisting of three transceivers (RF transceivers 1414-1, 1414-2, and 1414-3) and one active beacon (1418) as shown in FIG. 14, where the distance between each of the transceivers is fixed and known, the transmission/re-transmission information associated with each transceiver and the active beacon provides a known distance between each transceiver (RF transceivers 1414-1, 1414-2, and 1414-3) and the active beacon (1418), calculated using the known speed of RF signals. Accordingly, the control device 12 can be configured (e.g., programmed) to recognize that there are three different triangles formed between any two pairs of the transceivers and the active beacon (i.e., the triangles formed with the vertices: (1414-1, 1414-2, 1418), (1414-1, 1414-3, 1418), and (1414-2, 1414-2, 1418)). Based upon this information, trilateration techniques can be used to determine the position of the active beacon 1418 relative to the constellation configuration in three-dimensional space.

For example, through the above-described technique, or using any method known to one of ordinary skill in the art, distances, 1418X, 1418Y, and 1418Z can be determined relative to an axis-system 1401, 1402, and 1403 fixed relative to the constellation configuration associated with the RF transceivers (RF transceivers 1414-1, 1414-2, and 1414-3).

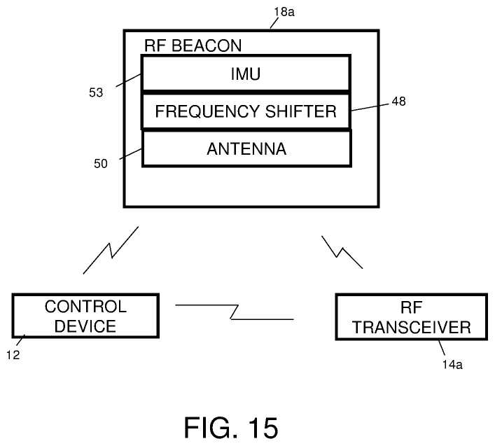

Referring now to FIG. 15, in an aspect, the system for alerting the medical provider of a possible displacement of the beacon includes a beacon 18a, a control device 12, and a transceiver 14a. The beacon 18a includes sensors (for example, but not limited to, an IMU 53 and a frequency shifter 48) and at least one antenna 50 that exchanges signals with the transceiver 14a, as described herein. In an aspect, the IMU 53 provides data to the control device 12 using a wireless communications protocol as described herein with respect to the accelerometer 52 (FIG. 2). The frequency shifter 48 responds to RF signals from the transceiver 14a, and this response informs the control device 12 of the position of the beacon. The frequency shifter 48 can be used to correct any drift error of the IMU 53, thereby increasing the positional accuracy of the IMU data. In an aspect, multiple frequency shifters 48 are used to obtain realtime calibration of the IMU 53.

Continuing to refer to FIG. 15, the IMU 53 and the frequency shifter 48 are used in a coordinated way to alert the medical provider if the beacon has been displaced in a way that might impact the medical procedure. To enable such an alert, the IMU 53 and frequency shifter 48 are registered to the same coordinate frame, and the IMU 53 is calibrating, possibly continuously, by the control device 12 which uses the frequency shifter data to correct for the IMU drift. The substantially continuous calibration happens when the IMU 53 experiences low acceleration. In an aspect, a calibration at this time is a matter of calibrating the gyroscope as if the IMU 53 were not moving, i.e. each of the three axes of the gyroscope should read approximately 0°/sec. At this time, the accelerometer alone can be calibrated to 0-2g. At a time when the IMU is experiencing high gravity, the system of the present teachings determines whether to notify the user that a potential impact to the accuracy of the IMU measurement has occurred. In operation, in an aspect, as the beacon moves, when an anomalous correlation between the change over time of the IMU position data (of the beacon) and the frequency shifter position data (of the beacon) is detected, and the beacon has undergone a gravity/inertial moment (according to the IMU data) that exceeds a pre-selected threshold, the medical provider is alerted that the beacon's position might have shifted enough to impact the medical procedure. When the anomalous correlation is detected, but the beacon's high gravity/inertial moment has not exceeded the pre-selected threshold, the IMU is recalibrated according to the frequency shifter data. In an aspect, high gravity is 2-4g. Operationally, the coordinate system of the IMU 53 and the frequency shifter 48 is recalibrated regularly, for example, but not limited to, every two minutes. When the device is purposely moved, calibration automatically happens when the device is repositioned. When the device is accidently moved, a check, either automatic, or manual, is performed on the system. In an aspect, the manual check is performed by locating a pre-selected spot on the medical object, for example, the patient, with a paired pointer probe. The pointer probe includes a tip whose location is known relative to the device.

Continuing to refer to FIG. 15, the alert can be in the form of a warning indicator on a computerized device having a user interface screen. In an aspect, the computerized device is, for example, but not limited to, a handheld device, a workstation, or a tablet. In an aspect, multiple beacons can be used during the medical procedure to track various anatomical features independently. In an aspect, the frequency shifter can be replaced and/or augmented by any positional tracking modality, for example, but not limited to, LIDAR and optical devices.

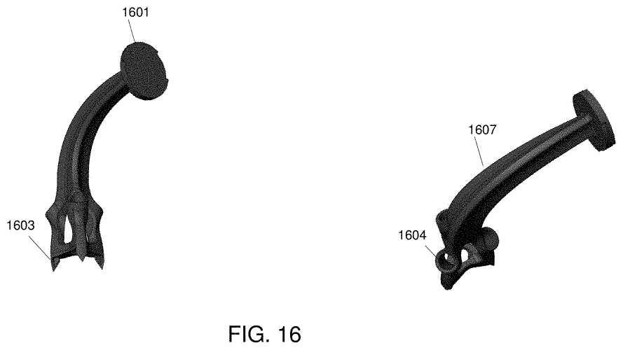

Referring now to FIG. 16, in a first configuration, a beacon interface 1601 is shown upon which a removable beacon is positioned. The removable beacon can be attached to the beacon interface 1601 with, for example, but not limited to, clips, VELCRO®, adhesive, and/or fasteners. In an aspect, the beacon can be fixedly attached to the arm. For example, the beacon and arm can be printed, molded, or otherwise fabricated as one piece. In an aspect, the beacon, the arm 1607, and the mounting anchor 1603 can be printed, molded, or otherwise fabricated as one piece. In an aspect, the mounting anchor 1603 is separate and distinct from the arm 1607. In such a configuration, the anchor 1603 is mounted to the anatomical feature, and the arm is then mounted onto the anchor 1603. The configuration in FIG. 16 includes cavities 1604 (for access to a fastening device) to enable further fastening and stabilization of the arm onto the anatomical feature.

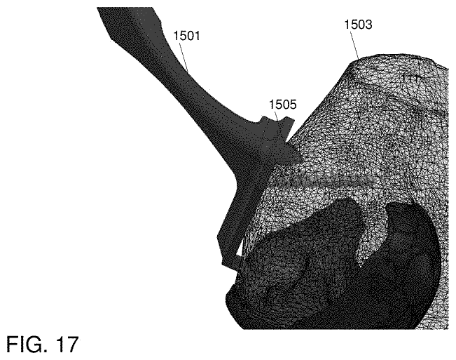

Referring now to FIG. 17, a further configuration of the arm and anchor are shown. In this configuration, arm 1501 is mounted or fabricated in a relatively flush position onto anchor 1505, which is shown attached to anatomical feature 1503.

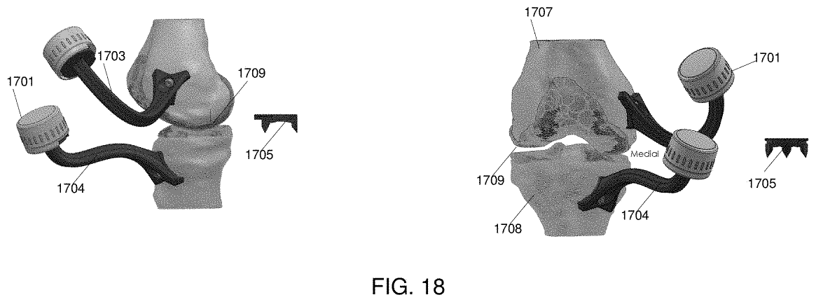

Referring now to FIG. 18, a beacon 1701 is attached to an arm 1703/1704 that itself is attached to a medical object anchor 1705, or the various components are fabricated as one or two pieces. In an aspect, the beacon 1701 can removably attach to the arm 1703/1704 to enable easy removal and replacement. The arms 1703/1704 are shaped to provide conformity to the contours of an anatomical feature when optimally positioned (on the anchor 1705). In an aspect, the arm is constructed of rigid material that maintains the beacon at a desired position during a medical procedure requiring the information provided by the sensors in the beacon. In an aspect, the arm is an extrusion of a desired length, shape, and thickness. In an aspect, the arm is fabricated from multiple sections that can interlock with each other to form various shapes and sizes of arms. In an aspect, the interlocking mechanism can enable twisted and curved connections between the shapes. For example, FIG. 18 illustrates devices of the present teachings attached to the adjacent parts of the patellofemoral joint 1709, specifically the femur 1707 and the tibia 1708. Exemplary arms 1703/1704 are shaped to provide optimal opportunity for the beacon 1701 to transmit signals to the control device 12 (FIG. 15) and receive signals from the RF transceiver 14a (FIG. 15). Specifically, the arm 1703 is shaped according to the geometry of the femur 1707 and soft tissue normally associated with the femur 1707, while the arm 1704 is shaped according to the geometry of the tibia 1708 and soft tissue normally associated with the tibia 1708. Note that the placement of the anchors 1705 and the arms 1703/1704 is not limited to the bones forming the patellofemoral joint 1709, but can be mounted upon any anatomical feature.

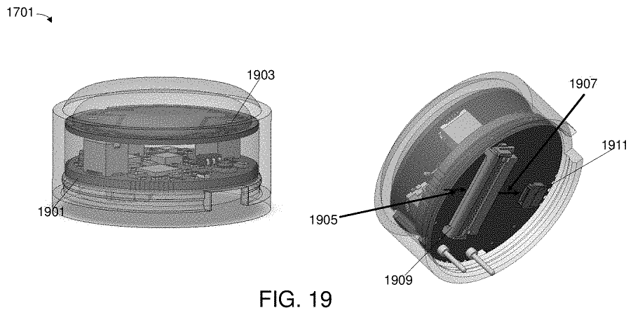

Referring now to FIG. 19, the beacon 1701 includes at least one RF frequency shifter and an IMU, similar to other configurations described herein. In an aspect, the beacon 1701 can include a power supply such as, for example, but not limited to, a battery (not shown), that can power the devices within the beacon 1701. In an aspect, the beacon 1701 can include connectors 1909/1911 that can electrically couple the devices within the beacon 1701 with a power supply that could be, for example, located within the arm 1703/1704 (FIG. 18). In an aspect, the connectors 1909/1911 slide 1905/1907 towards each other as the beacon 1701 couples with the arm 1703/1704 (FIG. 18). In an aspect, the power source can be the electrical grid and the wiring can provide a plug-in means of powering the beacon 1701. In an aspect, the beacon 1701 can be powered by continuous wireless power transfer. In an aspect, the beacon 1701 and/or the arm 1703/1704 (FIG. 18) include rechargeable batteries that can be charged wirelessly. In an aspect, both the beacon 1701 and the arm 1703/1704 (FIG. 18) can include power supply options, where one can back up the other in case of a primary power supply failure. In an aspect, the control device 12 receives an alert that the primary power supply has failed, and activates the backup power supply through communications means described herein. Other options for the beacon 1701 and power supplies and management for the beacon 1701 are contemplated by the present teachings.

Continuing to refer to FIG. 19, in an aspect, an RF device 1903, including frequency shifter 48 (FIG. 15), is mounted atop a control board 1901. On the control board 1901 is configured at least the IMU 53 (FIG. 15), and possibly communications means such as Bluetooth or wifi that enable communications between the beacon 1701 and the control device 12 (FIG. 15). Other sensors and board configurations are contemplated by the present teachings. For example, the RF device 1903 could be mounted below or beside the control board 1901, or could surround the control board 1901. The control board 1901 can include a processor that could be used to perform some or all of the system functions, including those that can also be performed by the control device 12.

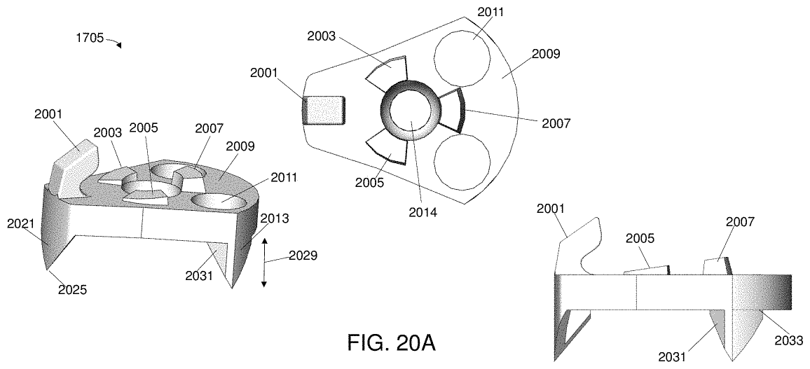

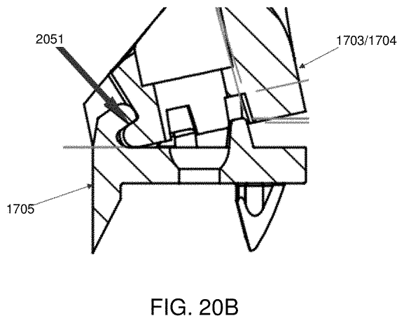

Referring now to FIGS. 20A and 20B, in an aspect, the mounting anchor 1705 operably couples the arm 1703/1704 (FIG. 18) with a medical object, such as, for example, but not limited to, an anatomical feature. In an aspect, the anchor 1705 attaches to the medical object by means of, for example, teeth 2013 that bite into the medical object and resist twisting and disengagement. In an aspect, each tooth includes a first section 2021 that follows the line 2033 of the anchor at a first end 2013 and tapers to a point 2025, the first section 2021 taking the shape of, for example, but not limited to, a triangle. In an aspect, the teeth 2013 are each 3-7 mm in length 2029. When mounting the anchor 1705, the point or tip 2025 engages with the mounting surface and enters the mounting surface after pressure is applied to the anchor 1705. In an aspect, some or all of the teeth 2013 are operably coupled with second sections 2031 that are positioned, for example, but not limited to, perpendicular to the first section 2021. The second sections 2031 provide further latching and stability to the anchor 1705, and therefore to the arm 1703/1704 (FIG. 18) and beacon 1701 (FIG. 18). The teeth 2013 are individually attached to the medical object by bone insertion techniques such as, for example, but not limited to, providing pressure or impacts to the anchor 1705.

Continuing to refer to FIGS. 20A and 20B, in an aspect, the anchor 1705 includes a coupling component 2001 and alignment features 2003/2005/2007. The coupling component 2001 can take any shape, including a mostly-closed or mostly-open hook-like geometry. In an aspect, the coupling component 2001 engages with the arm 1703/1704 for appropriate coupling integrity. In an aspect, the arm 1703/1704 includes a feature that accommodates the coupling component 2001, if necessary. In an aspect, the alignment features 2003/2005/2007 mate with cooperating features at the base of arm 1703/1704. In an aspect, the alignment features 2003/2005/2007 are configured to enable a hook and clocking mechanism. The hook and alignment features enable a highly repeatable removal and replacement of the arm. In an aspect, the faces of the alignment features 2003/2005/2007 are configured to be adjacent to the base of arm 1703/1704 (FIG. 18). Specifically, they are slanted and of varying, but gradually increasing, heights with respect to each other. These components enable correctly aligned coupling with the arm 1703/1704 (FIG. 18). Other types of alignment and coupling features and components are contemplated by the present teachings. For example, the anchor 1705 can include notched features that would couple with like features present on the arm 1703/1704 (FIG. 18). In an aspect, alignment arrows could enable correct alignment between the arm 1703/1704 (FIG. 18) and the anchor 1704.

Continuing to refer to FIGS. 20A and 20B, in an aspect, the anchor 1705 includes a relatively flat side 2009 that is configured to be adjacent to the base of the arm 1703/1704 (FIG. 18). Adjacent side 2009 provides a surface for providing pressure on the anchor 1705, the pressure causing the anchor 1705 to penetrate a mounting surface. In an aspect, the anchor 1705 includes coupling features that further ensure the integrity of the coupling between the arm 1703/1704 (FIG. 18) and the anchor 1705. In an aspect, the arm 1703/1704 (FIG. 18) is removably or fixedly attached at a first end to the beacon 1701 (FIG. 19), and removably or fixedly attached to the anchor 1705 at a second end through coupling 2051 (FIG. 20B) and an associated clocking feature enabled by the varying heights of features 2003/2005/2007. Attachment means include, for example, but not limited to, hooks and/or magnets and/or fasteners such as screws. In an aspect, magnets are positioned to stabilize the coupling. For example, anchor 1705 can include magnetic features 2011 that cooperatively engage with magnetic features (not shown) on the base of arm 1703/1704 (FIG. 18). In an aspect, the anchor includes a cavity 2014. The cavity 2014 provides a conduit to accommodate fasteners that further secure the arm 1703/1704 (FIG. 18) to the mounting surface. In an aspect, a fastener, such as a screw, can travel from the arm 1703/1704 (FIG. 18), through the anchor 1705, and into the mounting surface.

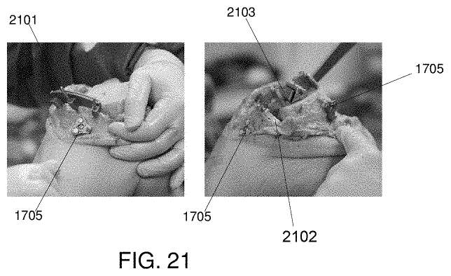

Referring now to FIG. 21, shown are anchors 1705 mounted upon the femur and the tibia alongside surgery assistive devices. Anchors 1705 operate cooperatively with equipment used to assist medical professionals in performing the surgery. For example, shown are a spreader 2101 and a femoral cutting block 2103. Also shown is a universal cutting block 2101 that can be used for operations with, for example, a tibia or a femur. Also shown is a tibial trial implant/insert 2102. The device of the present teachings does not interfere with common surgical device placement because of its modest footprint.

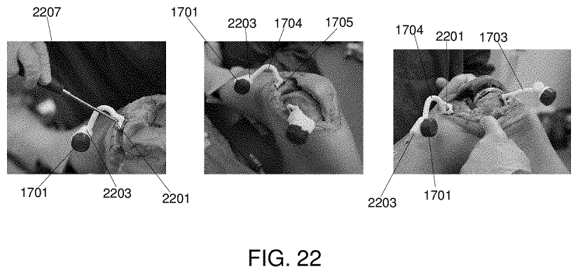

Referring now to FIG. 22, shown are arm/anchor 1703/1705 mounted upon the femur and arm/anchor 1704/1705 mounted upon the tibia. An exemplary power supply 2203 for the beacon 1701 is shown. Other possible power options have been described herein. In an aspect, securing the arm 1704 to the tibia includes using tool 2207 to tighten the fastener 2201 that traverses both the arm 1703/1704 and the anchor 1705. In an aspect, connection can be accomplished toollessly, for example, by use of wing nut fasteners. In various aspects, the fastener 2201 includes a screw or multiple screws, screws and adhesive, or clamps. In an aspect, no top-entry fastener is present.

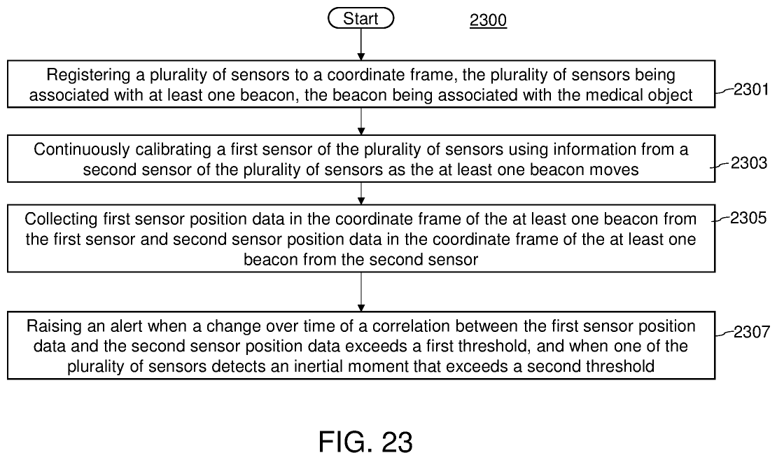

Referring now to FIG. 23, method 2300 for alerting a medical professional if a medical object has moved during a medical procedure includes, but is not limited to including, registering 2301 a plurality of sensors to a coordinate frame, the plurality of sensors being associated with at least one beacon, the beacon being associated with the medical object, calibrating 2303, possibly continuously, a first sensor of the plurality of sensors using information from a second sensor of the plurality of sensors as the at least one beacon moves, collecting 2305 first sensor position data in the coordinate frame of the at least one beacon from the first sensor and second sensor position data in the coordinate frame of the at least one beacon from the second sensor, and raising 2307 an alert when a change over time of a correlation between the first sensor position data and the second sensor position data exceeds a first threshold, and when one of the plurality of sensors detects an inertial moment that exceeds a second threshold. In an aspect, the at least one beacon includes a power supply configured to power the plurality of sensors. In an aspect, the power supply includes at least one battery housed in a battery storage area. In an aspect, the first sensor includes an inertial measurement unit (IMU) configured to provide IMU beacon location data and the inertial moment of the at least one beacon to at least one controller. In an aspect, the second sensor includes a frequency shifter configured to receive a signal from at least one RF transceiver and provide a signal to at least one controller. Method 2300 can optionally include determining a beacon location of the at least one beacon using range-Doppler processing, receiving data from the first sensor wirelessly, correcting drift error of the first sensor using the second sensor, and substantially continuously calibrating the first sensor based on information from another second sensors. Method 2300 can be executed by a controller that is local or remote to the device.

As will be appreciated by one of skill in the art, the concepts described herein may be embodied as a method, data processing system, computer program product and/or computer storage media storing an executable computer program. Accordingly, the concepts described herein may take the form of an entirely hardware embodiment, an entirely software embodiment or an embodiment combining software and hardware aspects all generally referred to herein as a “circuit” or “module.” Any process, step, action and/or functionality described herein may be performed by, and/or associated to, a corresponding module, which may be implemented in software and/or firmware and/or hardware. Furthermore, the disclosure may take the form of a computer program product on a tangible computer usable storage medium having computer program code embodied in the medium that can be executed by a computer. Any suitable tangible computer readable medium may be utilized including hard disks, CD-ROMs, electronic storage devices, optical storage devices, or magnetic storage devices.

Some embodiments are described herein with reference to flowchart illustrations and/or block diagrams of methods, systems and computer program products. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer (to thereby create a special purpose computer), special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable memory or storage medium that can direct a computer or other programmable data processing apparatus to function in a particular manner, such that the instructions stored in the computer readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

It is to be understood that the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved. Although some of the diagrams include arrows on communication paths to show a primary direction of communication, it is to be understood that communication may occur in the opposite direction to the depicted arrows.

Computer program code for carrying out operations of the concepts described herein may be written in an object-oriented programming language such as Java® or C++. However, the computer program code for carrying out operations of the disclosure may also be written in conventional procedural programming languages, such as the “C” programming language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the embodiments described herein are not limited to what has been particularly shown and described herein above. In addition, unless mention was made above to the contrary, it should be noted that all of the accompanying drawings are not to scale. A variety of modifications and variations are possible in light of the above teachings.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method for detecting possibly inaccurate information associated with a medical object during surgery comprising:
registering a plurality of sensors to a coordinate frame, the plurality of sensors being associated with at least one beacon, the at least one beacon being associated with the medical object;
calibrating a first sensor of the plurality of sensors using information from a second sensor of the plurality of sensors as the at least one beacon moves;
collecting first sensor position data in the coordinate frame of the at least one beacon from the first sensor and second sensor position data in the coordinate frame of the at least one beacon from the second sensor; and
raising an alert when a change over time of a correlation between the first sensor position data and the second sensor position data exceeds a first threshold, and when one of the plurality of sensors detects an inertial moment that exceeds a second threshold.

2. The method as in claim 1 wherein the at least one beacon comprises:
a power supply configured to power the plurality of sensors.

3. The method as in claim 2 wherein the calibrating comprises:
continuous calibration.

4. The method as in claim 1 wherein the first sensor comprises:
an inertial measurement unit (IMU) configured to provide IMU beacon location data and the inertial moment of the at least one beacon to at least one controller.

5. The method as in claim 1 wherein the second sensor comprises:
a frequency shifter configured to receive a signal from an RF transceiver and to provide the signal to at least one controller.

6. The method of claim 1 further comprising:
determining a beacon location of the at least one beacon using range-Doppler processing.

7. The method as in claim 1 further comprising:
receiving data from the first sensor wirelessly.

8. The method as in claim 1 further comprising:
correcting drift error of the first sensor using the second sensor.

9. The method as in claim 1 further comprising:
calibrating the first sensor based on information from another second sensor.

10. A system for detecting possible inaccurate information associated with a medical object during surgery comprising:
at least one beacon including a plurality of sensors;
an anchor configured to operably couple with the medical object;
an arm having a first end and a second end, the first end configured to operably couple with the at least one beacon, the second end configured to operably couple with the anchor; and
at least one controller communicatively coupled with at least one frequency shifter, the at least one frequency shifter being configured to transmit a first signal to the at least one controller and receive a second signal from at least one transceiver, the at least one controller configured to execute instructions including:
registering the plurality of sensors to a coordinate frame, the plurality of sensors being associated with the at least one beacon, the at least one beacon being associated with the medical object;
calibrating a first sensor of the plurality of sensors, the calibrating using information from a second sensor of the plurality of sensors as the at least one beacon moves;
collecting first sensor position data in the coordinate frame of the at least one beacon from the first sensor and second sensor position data in the coordinate frame of the at least one beacon from the second sensor; and
raising an alert if a change over time of a correlation between the first sensor position data and the second sensor position data exceeds a first threshold, and if one of the plurality of sensors detects an inertial moment that exceeds a second threshold.

11. The system as in claim 10 wherein the at least one beacon comprises:
a power supply configured to power the plurality of sensors.

12. The system as in claim 10 wherein the arm comprises:
an anatomically-consistent geometric shape.

13. The system as in claim 10 wherein the first sensor comprises:
an inertial measurement unit (IMU) configured to provide IMU beacon location data and the inertial moment of the at least one beacon to the at least one controller.

14. The system as in claim 10 wherein the second sensor comprises:
a positional tracking modality.

15. The system as in claim 10 wherein the at least one controller is communicative coupled with the at least one transceiver.

16. The system as in claim 10 wherein the anchor comprises:
a plurality of teeth configured to operably connect the anchor with the medical object.

17. The system as in claim 16 wherein the plurality of teeth comprises:
a plurality of triangle-shaped forms operably coupled with the anchor; and
a plurality of components orthogonally coupled with the plurality of triangle-shaped forms.

18. The system of claim 10 wherein the at least one controller is configured to use range-Doppler processing to determine a location of the at least one beacon.

19. The system as in claim 10 wherein the alert is a warning indicator on a computerized device having a user interface screen.

20. The system as in claim 19 wherein the computerized device is a laptop computer device, a handheld computer device, a desktop computer device, or a tablet computer device.
